(19) United States
(12) Patent Application Publication
(10) Pub. No.: US 2020/0024246 A1
(43) Pub. Date: Jan. 23, 2020

(54) POGOSTONE DERIVATIVES AND METHODS OF USING THE SAME

(71) Applicant: Hamyard Associates LLC, Winter Park, FL (US)

(72) Inventors: George A. Kraus, Ames, IA (US); Joel R. Coats, Ames, IA (US); Edmund J. Norris, Ames, IA (US)

(73) Assignee: Hamyard Associates LLC, Winter Park, FL (US)

(21) Appl. No.: 16/518,550

(22) Filed: Jul. 22, 2019

Related U.S. Application Data
(60) Provisional application No. 62/702,158, filed on Jul. 23, 2018.

(51) Int. Cl. C07D 309/38; C07D 493/04; C07D 215/20; C07C 49/788; C07C 49/248; C07D 311/56; C07D 407/06; C07C 49/203; C07D 233/96; C07D 413/06; A01N 43/80; A01N 43/50; A01N 43/16; A01N 31/16; A01N 43/28

(52) U.S. Cl. CPC C07D 309/38; A01N 43/28; C07D 215/20; C07C 49/788; C07C 49/248; C07D 311/56; C07D 407/06; C07C 49/203; C07D 233/96; C07D 413/06; A01N 43/80; A01N 43/50; A01N 43/16; A01N 31/16; C07D 493/04

(57) ABSTRACT

Described are novel derivatives of pogostone and methods of using the same.

### RELATED APPLICATIONS

This application claims priority under 35 U.S.C. § 119(e) to U.S. Provisional Application 62/702,158 filed Jul. 23, 2018, the entire contents of which are incorporated herein by reference.

### FIELD

The present disclosure relates generally to derivatives of pogostone, as well as methods of using the disclosed derivatives as insecticides, repellents, larvicides, fungicides, anti-microbials, antibiotics, and/or herbicides.

### BACKGROUND

The following discussion is merely provided to aid the reader in understanding the disclosure and is not admitted to describe or constitute prior art thereto.

Synthetic insecticides have been immensely important in combatting dangerous and/or destructive arthropods, such as mosquitoes, throughout the world. By promoting the continued influx of sodium ions across the membrane of neurons in arthropods, pyrethroid insecticides cause hyper-excitability of the arthropod nervous system, contributing to spastic paralysis and inevitably death. Because pyrethroids readily migrate across the arthropod cuticle and diffuse evenly throughout the arthropod, they exert their toxic effects minutes after the target arthropod comes into contact with these compounds. One important symptom of pyrethroid intoxication is rapid immobilization, also known as “knockdown.” Unlike some slow acting insecticides, pyrethroids are ideal for public health vector control because this knockdown effect prevents mosquitoes from feeding on future hosts. Moreover, it likely contributes to the mortality of the intoxicated arthropod through numerous mechanisms, for example, desiccation, susceptibility to predation, and the inhibition of grooming which mitigates the accumulation of fungal spores on the arthropod. This knockdown effect is an important consideration in the development of future insecticidal formulations that contain natural products with novel modes of action.

One of the hurdles to identifying new insecticidal formulations for the control of pest populations is ensuring that these new mixtures will be fast-acting and cause high mortality while simultaneously being safe for application in the environment. Although many toxic synthetic insecticides exist which cause rapid nervous system intoxication, their high toxicity to humans and other vertebrates and relatively long half-lives in the environment prevents them from being viable avenues to follow in the search for new insecticides. It is paramount to identify candidate insecticides which have low mammalian toxicity, degrade rapidly, and still quickly immobilize and kill mosquitoes and other pests. Because of the rapid development of resistance to many of the currently available insecticides on the market, the continual development of new insecticides is the only means available to continue to control pest arthropod populations.

Thus, there remains a need for effective agents that can safely be used as insecticides and arthropod repellents/knockdown agents, as well as a need for similarly same and effective larvicides, fungicides, anti-microbials, antibiotics, and/or herbicides. The present disclosure fulfills this need.

### SUMMARY

Described herein are novel pogostone derivatives and methods of using the same as insecticides, repellents, larvicides, fungicides, anti-microbials, antibiotics, and/or herbicides.

In one aspect, the present disclosure provides pogostone derivatives or salts thereof comprising the structure:

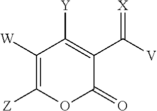

wherein V is selected from a group consisting of a substituted or unsubstituted C₁-C₁₂ unbranched or branched alkyl, substituted or unsubstituted C₂-C₁₂ unbranched or branched alkenyl, substituted or unsubstituted C₃-C₁₂ unbranched or branched alkynyl, substituted or unsubstituted C₃-C₁₂ unbranched or branched cycloalkyl, substituted or unsubstituted heteroaryl, and a substituted or unsubstituted C₃-C₁₂ unbranched or branched cycloalkenyl; wherein W comprises a hydrogen or a substituted or unsubstituted C₃-C₆ saturated or unsaturated alkane or alkene; wherein X is selected from a group consisting of an oxygen and a sulfur atom; wherein Y is selected from a group consisting of a hydroxyl group, sulphhydryl, amino, a halogen, and an ether with variable alkyl length (C₁-C₇); wherein Z comprises a substituted or unsubstituted C₁-C₁₂ unbranched or branched alkyl, substituted or unsubstituted C₃-C₁₂ unbranched or branched cycloalkyl, substituted or unsubstituted heteroaryl, and substituted or unsubstituted C₃-C₁₂ unbranched or branched cycloalkenyl; and wherein W may be linked to Z via a substituted or unsubstituted C₃-C₆ saturated or unsaturated alkane or alkene.

In another aspect, the present disclosure provides compounds or salts thereof of the following formula:

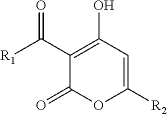

wherein R₁ is selected from a group consisting of a substituted or unsubstituted C₁-C₁₂ unbranched or branched alkyl, substituted or unsubstituted C₂-C₁₂ unbranched or branched alkenyl, substituted or unsubstituted C₃-C₁₂ unbranched or branched alkynyl, substituted or unsubstituted C₃-C₁₂ unbranched or branched cycloalkyl, substituted or unsubstituted heteroaryl, and a substituted or unsubstituted C₃-C₁₂ unbranched or branched cycloalkenyl; and wherein R₂ is selected from a group consisting of a hydrogen, a substituted or unsubstituted C₃-C₁₂ unbranched or branched alkyl, substituted or unsubstituted C₂-C₁₂ unbranched or branched alkenyl, substituted or unsubstituted C₃-C₁₂ unbranched or branched alkynyl, substituted or unsubstituted C₃-C₁₂ unbranched or branched cycloalkyl, substituted or unsubstituted heteroaryl, and substituted or unsubstituted C₃-C₁₂ unbranched or branched cycloalkenyl, a hydroxyl group, sulphhydryl, amino, a halogen, and an ether with variable alkyl length (C₁-C₇).

In some embodiments, R₁ may be a substituted or unsubstituted C₁-C₁₂ unbranched or branched alkyl. In some embodiments, R₂ may be a methyl group, while in some embodiments, R₂ may comprise a unsubstituted C₃-C₁₂ unbranched or branched cycloalkyl.

In some embodiments, the compound may have the structure of Formula 4, Formula 5, or Formula 9.

In another aspect, the present disclosure provides compounds or salts thereof of the following formula:

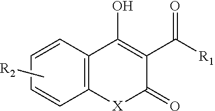

wherein R₁ is selected from a group consisting of a substituted or unsubstituted C₁-C₁₂ unbranched or branched alkyl, substituted or unsubstituted C₂-C₁₂ unbranched or branched alkenyl, substituted or unsubstituted C₃-C₁₂ unbranched or branched alkynyl, substituted or unsubstituted C₃-C₁₂ unbranched or branched cycloalkyl, substituted or unsubstituted heteroaryl, and a substituted or unsubstituted C₃-C₁₂ unbranched or branched cycloalkenyl; wherein R₂ is selected from a group consisting of a hydrogen, a substituted or unsubstituted C₃-C₁₂ unbranched or branched alkyl, substituted or unsubstituted C₂-C₁₂ unbranched or branched alkenyl, substituted or unsubstituted C₃-C₁₂ unbranched or branched alkynyl, substituted or unsubstituted C₃-C₁₂ unbranched or branched cycloalkyl, substituted or unsubstituted heteroaryl, and substituted or unsubstituted C₃-C₁₂ unbranched or branched cycloalkenyl, a hydroxyl group, sulphhydryl, amino, a halogen, and an ether with variable alkyl length (C₁-C₇); and wherein X is selected from a group consisting of an oxygen, a carbon, and a nitrogen.

In some embodiments, R₁ may be a substituted or unsubstituted C₁-C₁₂ unbranched or branched alkyl. In some embodiments, R₂ may be a hydrogen. In some embodiments, X is an oxygen, while is some embodiments, X is a nitrogen.

In some embodiments, the compound may have the structure of Formula 13, Formula 14, Formula 16, Formula 17, Formula 19, Formula 20, Formula 21, Formula 22, Formula 23, Formula 24, or Formula 27. In more particular embodiments, the compound may have the structure of Formula 13. In other embodiments, the compound may have the structure of Formula 36, Formula 37, Formula 38, Formula 39, Formula 40, or Formula 41.

In another aspect, the present disclosure provides compounds or salts thereof of the following formula:

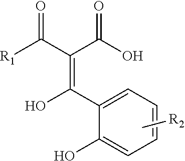

wherein R₁ is selected from a group consisting of a substituted or unsubstituted C₁-C₁₂ unbranched or branched alkyl, substituted or unsubstituted C₂-C₁₂ unbranched or branched alkenyl, substituted or unsubstituted C₃-C₁₂ unbranched or branched alkynyl, substituted or unsubstituted C₃-C₁₂ unbranched or branched cycloalkyl, substituted or unsubstituted heteroaryl, and a substituted or unsubstituted C₃-C₁₂ unbranched or branched cycloalkenyl; and wherein R₂ is selected from a group consisting of a hydrogen, a substituted or unsubstituted C₃-C₁₂ unbranched or branched alkyl, substituted or unsubstituted C₂-C₁₂ unbranched or branched alkenyl, substituted or unsubstituted C₃-C₁₂ unbranched or branched alkynyl, substituted or unsubstituted C₃-C₁₂ unbranched or branched cycloalkyl, substituted or unsubstituted heteroaryl, and substituted or unsubstituted C₃-C₁₂ unbranched or branched cycloalkenyl, a hydroxyl group, sulphhydryl, amino, a halogen, and an ether with variable alkyl length (C₁-C₇).

In yet another embodiment, the present disclosure provides a compound or salt having the structure of Formula 28.

With respect to all of the foregoing aspects, in some embodiments the structure of the pogostone derivative or salt thereof may be selected from a group consisting of Formula 1, Formula 2, Formula 3, Formula 4, Formula 5, Formula 6, Formula 7, Formula 8, Formula 9, Formula 10, Formula 11, Formula 12, Formula 13, Formula 14, Formula 15, Formula 16, Formula 17, Formula 18, Formula 19, Formula 20, Formula 21, Formula 22, Formula 23, Formula 24, Formula 25, Formula 26, Formula 27, Formula 28, Formula 29, Formula 30, Formula 31, Formula 32, Formula 33, Formula 34, Formula 35, Formula 36, Formula 37, Formula 38, Formula 39, Formula 40, and Formula 41. More specifically, in some embodiments, the structure of the pogostone derivative or salt thereof may be selected from a group consisting of Formula 1, Formula 2, Formula 3, Formula 4, Formula 5, Formula 9, Formula 13, Formula 14, Formula 15, Formula 16, Formula 17, Formula 19, Formula 20, Formula 21, Formula 22, Formula 23, Formula 24, Formula 27, Formula 29, Formula 30, Formula 31, Formula 32, Formula 33, Formula 34, Formula 35, and Formula 36.

In one aspect, the present disclosure provides pogostone derivatives or salts thereof having a structure selected from the group consisting of Formula 36, Formula 37, Formula 38, Formula 39, Formula 40, Formula 41, Formula 42, Formula 43, Formula 44, Formula 45, Formula 46, Formula 47, and Formula 48.

In one aspect, the present disclosure provides pogostone derivatives or salts thereof having a structure selected from the group consisting of Formula A3, Formula B3, Formula C3, Formula D3, Formula C5, Formula C6, Formula C7, Formula C8, Formula C9, Formula C10, Formula C11, Formula C12, Formula C13, Formula C14, Formula C15, Formula C16, Formula C17, Formula C18, Formula C19, Formula C20, Formula C21, Formula C22, Formula C23, Formula C24, Formula C25, Formula C26, Formula C27, Formula N1, Formula N2, Formula N3, Formula N4, Formula N5, Formula N6, Formula N7, Formula N8, Formula N9, Formula N10, AND Formula N11. In some embodiments, the compound or salt thereof is selected from the group consisting of Formula C10, Formula C15, Formula C19, Formula C24, Formula C25, and Formula C26.

In some embodiments of any of the foregoing aspects, the compound kills or repels arthropods belonging to the class of Insecta, Acarina, Chilognatha, Epimorpha, or Isopoda.

In some embodiments of any of the foregoing aspects, the compound is a single enantiomer or a diastereomer.

In some embodiments of any of the foregoing aspects, the compound is a racemic mixture or a diastereomer.

In another aspect, the present disclosure provides insecticidal, antibacterial, fungicidal, or herbicidal compositions comprising a compound according to any one of the foregoing aspects or embodiments and an acceptable vehicle.

In some embodiments, the carrier may be a solid, a liquid, or a gas. In some embodiments, the composition may be formulated as a lotion, spray, aerosol, or cream. In some embodiments, composition is formulated as a fragrance, perfume, or cologne.

The present disclosure also provides a pogostone derivative according to any one of the foregoing aspects or embodiments for use as an insecticide, larvicide, fungicide, anti-microbial, antibiotic, and/or herbicide.

The present disclosure also provides a use of a pogostone derivative according to any one of the foregoing aspects or embodiments as an insecticide, larvicide, fungicide, anti-microbial, antibiotic, and/or herbicide.

In another aspect, the present disclosure provides methods of killing or repelling arthropods comprising applying to a target area an effective amount of a compound or salt thereof selected from the group consisting of:

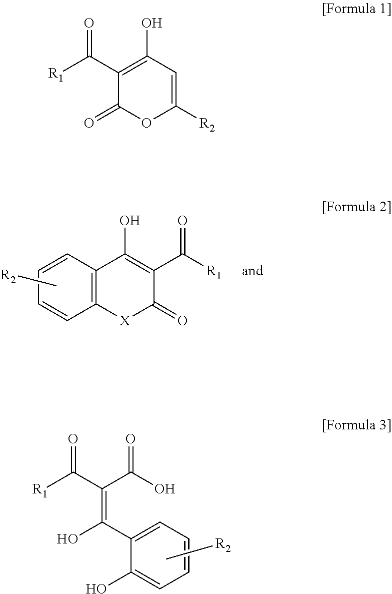

wherein R₁ is selected from a group consisting of a substituted or unsubstituted C₁-C₁₂ unbranched or branched alkyl, substituted or unsubstituted C₂-C₁₂ unbranched or branched alkenyl, substituted or unsubstituted C₃-C₁₂ unbranched or branched alkynyl, substituted or unsubstituted C₃-C₁₂ unbranched or branched cycloalkyl, substituted or unsubstituted heteroaryl, and a substituted or unsubstituted C₃-C₁₂ unbranched or branched cycloalkenyl; wherein R₂ is selected from a group consisting of a hydrogen, a substituted or unsubstituted C₃-C₁₂ unbranched or branched alkyl, substituted or unsubstituted C₂-C₁₂ unbranched or branched alkenyl, substituted or unsubstituted C₃-C₁₂ unbranched or branched alkynyl, substituted or unsubstituted C₃-C₁₂ unbranched or branched cycloalkyl, substituted or unsubstituted heteroaryl, and substituted or unsubstituted C₃-C₁₂ unbranched or branched cycloalkenyl, a hydroxyl group, sulphhydryl, amino, a halogen, and an ether with variable alkyl length (C₁-C₇); and wherein X is selected from a group consisting of an oxygen, a carbon, or a nitrogen.

In some embodiments, the compound may have a structure selected from the group consisting of Formula 4, Formula 5, Formula 9, Formula 13, Formula 14, Formula 16, Formula 17, Formula 19, Formula 20, Formula 21, Formula 22, Formula 23, Formula 24, Formula 27, Formula 28, Formula 29, Formula 30, Formula 31, Formula 32, Formula 33, Formula 34, Formula 35, Formula 36, Formula 36, Formula 37, Formula 38, Formula 39, Formula 40, and Formula 41. More specifically, the compound may have the structure of Formula 13.

In another aspect, the present disclosure provides methods of killing or repelling arthropods comprising applying to a target area an effective amount of a compound or salt thereof selected from the group consisting of Formula 36, Formula 37, Formula 38, Formula 39, Formula 40, Formula 41, Formula 42, Formula 43, Formula 44, Formula 45, Formula 46, Formula 47, and Formula 48.

In another aspect, the present disclosure provides methods of killing or repelling arthropods comprising applying to a target area an effective amount of a compound or salt thereof selected from the group consisting of Formula A3, Formula B3, Formula C3, Formula D3, Formula C5, Formula C6, Formula C7, Formula C8, Formula C9, Formula C10, Formula C11, Formula C12, Formula C13, Formula C14, Formula C15, Formula C16, Formula C17, Formula C18, Formula C19, Formula C20, Formula C21, Formula C22, Formula C23, Formula C24, Formula C25, Formula C26, Formula C27, Formula N1, Formula N2, Formula N3, Formula N4, Formula N5, Formula N6, Formula N7, Formula N8, Formula N9, Formula N10, and Formula N11. In some embodiments, the compound or salt thereof is selected from the group consisting of Formula C10, Formula C15, Formula C19, Formula C24, Formula C25, and Formula C26.

In some embodiments of the methods of killing or repelling arthropods, the target area comprises a plant, such as a crop plant, including but not limited to, corn, soybeans, wheat, vegetables, fruits, or cotton. In some embodiments, the target area is a seed of the plant.

In some embodiments of the methods of killing or repelling arthropods, applying the compound comprises spraying the target with the compound, while in some embodiments, applying comprises a vapor-delivery system.

In some embodiments of the methods of killing or repelling arthropods, the target area comprises an animal. For example, in some embodiments, the animal may be a livestock animal, a companion animal, or a human.

In some embodiments of the methods of killing or repelling arthropods, applying the compound comprises spraying the animal with a liquid or aerosol, while in some embodiment, applying comprises contacting the animal with a lotion, gel, cream, or balm comprising the compound.

In some embodiments of the methods of killing or repelling arthropods, the arthropod may be selected form the group consisting of mosquitos, ticks, fleas, ants, corn borers, grain borers, beetles, flies, and cockroaches. In some embodiments, the arthropod may be selected from the group consisting of blood-sucking insects, biting insects, cockroaches, mosquitoes, blackfly, fleas, house flies, barn fly, face fly, bush fly, deer fly, horse fly, gnats, beetle, beer bug, louse, bed bug, earwig, ant, aphid, spruce bud worm, corn borer, sand flea, tsetse fly, assassin bug, biting flies, sand fly, stored grain pests, clothes moths, ticks, mites, spiders, phytophagous pests, and hematophagous pests.

In a further aspect, the present disclosure provides, methods of killing or reducing the growth of undesirable vegetation or weeds comprising applying to a target area an effective amount of a compound or salt thereof selected from the group consisting of:

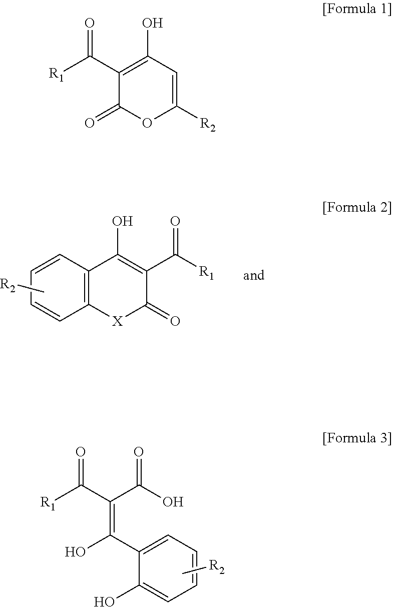

wherein R₁ is selected from a group consisting of a substituted or unsubstituted C₁-C₁₂ unbranched or branched alkyl, substituted or unsubstituted C₂-C₁₂ unbranched or branched alkenyl, substituted or unsubstituted C₃-C₁₂ unbranched or branched alkynyl, substituted or unsubstituted C₃-C₁₂ unbranched or branched cycloalkyl, substituted or unsubstituted heteroaryl, and a substituted or unsubstituted C₃-C₁₂ unbranched or branched cycloalkenyl; wherein R₂ is selected from a group consisting of a hydrogen, a substituted or unsubstituted C₃-C₁₂ unbranched or branched alkyl, substituted or unsubstituted C₂-C₁₂ unbranched or branched alkenyl, substituted or unsubstituted C₃-C₁₂ unbranched or branched alkynyl, substituted or unsubstituted C₃-C₁₂ unbranched or branched cycloalkyl, substituted or unsubstituted heteroaryl, and substituted or unsubstituted C₃-C₁₂ unbranched or branched cycloalkenyl, a hydroxyl group, sulphhydryl, amino, a halogen, and an ether with variable alkyl length (C₁-C₇); and wherein X is selected from a group consisting of an oxygen, a carbon, or a nitrogen.

In some embodiments, the compound may have a structure selected from the group consisting of Formula 4, Formula 5, Formula 9, Formula 13, Formula 14, Formula 16, Formula 17, Formula 19, Formula 20, Formula 21, Formula 22, Formula 23, Formula 24, Formula 27, Formula 28, Formula 29, Formula 30, Formula 31, Formula 32, Formula 33, Formula 34, Formula 35, Formula 36, Formula 36, Formula 37, Formula 38, Formula 39, Formula 40, and Formula 41. More specifically, the compound may have the structure of Formula 13.

In another aspect, the present disclosure provides methods of killing or reducing the growth of undesirable vegetation or weeds comprising applying to a target area an effective amount of a compound or salt thereof selected from the group consisting of Formula 36, Formula 37, Formula 38, Formula 39, Formula 40, Formula 41, Formula 42, Formula 43, Formula 44, Formula 45, Formula 46, Formula 47, and Formula 48.

In another aspect, the present disclosure provides methods of killing or reducing the growth of undesirable vegetation or weeds comprising applying to a target area an effective amount of a compound or salt thereof selected from the group consisting of Formula A3, Formula B3, Formula C3, Formula D3, Formula C5, Formula C6, Formula C7, Formula C8, Formula C9, Formula C10, Formula C11, Formula C12, Formula C13, Formula C14, Formula C15, Formula C16, Formula C17, Formula C18, Formula C19, Formula C20, Formula C21, Formula C22, Formula C23, Formula C24, Formula C25, Formula C26, Formula C27, Formula N1, Formula N2, Formula N3, Formula N4, Formula N5, Formula N6, Formula N7, Formula N8, Formula N9, Formula N10, AND Formula N11.

In some embodiments of the methods of killing or reducing the growth of undesirable vegetation or weeds, the undesirable vegetation comprises pigweed (Amaranthus retroflexus), poinsettia (Euphorbia heterophylla), nutsedge (Cyperus esculentus), morning glory (Ipomoea hederacea), or a combination thereof.

The foregoing general description and following detailed description are exemplary and explanatory and are intended to provide further explanation of the invention.

### BRIEF DESCRIPTION OF DRAWINGS

FIG. 1 shows the results of testing the insecticidal capacity of the compounds of Formula 13 and Formula 14 against insecticide-resistant mosquitoes. Pyrethroid-resistant strain of Aedes aegypti (Puerto Rican strain) were contacted with Formulas 13 and 14, and these compounds outperformed natural pyrethrins on this pyrethroid-resistant strain of mosquitoes.

FIG. 2 shows that seeds can be protected by the disclosed pogostone derivatives, and unlike pogostone, the derivatives do not cause phytotoxicity. Panel A shows the percentage survival of western corn rootworm exposed to various insecticides applied to seeds 1 week after introduction of larvae. Significant mortality was observed among seeds treated with pogostone and Formula 13. This percentage mortality was similar to seeds treated with various concentrations of imidacloprid, a neonicotinoid insecticide currently utilized as a seed treatment. Panel B shows the percentage survival of corn seeds exposed to various insecticides applied to seeds 1 week after introduction of larvae. Significant phytotoxicity was observed among seeds treated with pogostone but not its derivative, Formula 13.

FIG. 3 shows the results of spray-cup testing for several pogostone derivatives compared to thymol in various insects. Panel (a) shows the results of testing on house flies, panel (b) shows the results of testing on German roaches, and panel (c) shows the results of testing on maize weevils. Discrete concentrations of thymol (2.5% or 10% w/v) in water were compared to a single concentration of Formula 13 in water (2.5% w/v). The efficacy of Formula 13 at a lower concentration either surpassed the efficacy of thymol at both an equal or higher concentration when applied to (a) house flies and (b) German roaches. Formula 13 applied at 2.5% performed better than thymol applied at an equal concentration and as well as thymol applied in a 10% concentration against (c) maize weevils. This figure demonstrates that Formula 13 is more insecticidal than thymol against multiple pest arthropods and has the potential to be applied as an insecticidal spray, common for many commercial insecticidal formulations.

FIG. 4 shows the results of a leaf spray/dip test comparing the effectiveness of pogostone derivative Formula 13 against thymol in killing arthropods. Panel (a) shows that Formula 13 is significantly more effective at killing soybean aphids compared to thymol or a control, and panel (b) shows similar results against spider mites.

FIG. 5 shows the results of a house fly feeding test (choice assay), which indicates that house flies can be baited by the disclosed pogostone derivatives, several of which appear to be as toxic, if not more toxic, than boric acid (a commonly used fly bait).

FIG. 6 shows the combined effect of Formula 13 and chlorfenapyr applied topically to the pronotum of adult female Aedes aegypti at discrete doses. The dose of chlorfenapyr applied (75 ug/mL) and Formula 13 (500 ug/mL) corresponds to very low mortality at 24 hour, allowing synergism to be calculated. The percentage mortality of the combined solution was significantly higher than both chlorfenapyr applied alone or Formula 13 applied alone. This combined effect was higher than the effect expected from an additive model. This suggests that a synergistic interaction may account for these combined effects.

FIG. 7 shows the 1-hr percentage knockdown and 24-hr percentage mortality caused by topical applications of dibrom alone and in combination with Formula 13. Dibrom alone caused very little 24-hr mortality (11%) and Formula 13 alone produced similar 24-hr mortality (11.2%). When applied in combination, dibrom and Formula 13 caused 49.6% mortality at 24 hr after application, indicating strong synergism between these two components. No increase in 1-hr knockdown was observed in these mixtures compared to the insecticides applied by themselves (no synergism in this effect).

FIG. 8 shows the 1-hr percentage knockdown and 24-hr percentage mortality caused by topical applications of permethrin alone and in combination with Formula 13. Permethrin alone caused very little 24-hr mortality (28%) and Formula 13 alone produced similar 24-hr mortality (11.2%). When applied in combination, permethrin and Formula 13 caused 97.3% mortality at 24 hr after application, indicating strong synergism between these two components. No increase in 1-hr knockdown was observed in these mixtures compared to the insecticides applied by themselves (no synergism in this effect).

FIG. 9 shows the 1-hr percentage knockdown and 24-hr percentage mortality caused by topical applications of thiamethoxam alone and in combination with Formula 13. Thiamethoxam alone caused very little 24-hr mortality (25.6%) and Formula 13 alone produced similar 24-hr mortality (11.2%). When applied in combination, thiamethoxam and Formula 13 caused 93.3% mortality at 24 hr after application, indicating strong synergism between these two components. A significant increase in 1-hr knockdown was also observed in the mixture of these two insecticides compared to each applied by themselves.

FIG. 10 shows the efficacy of various synergists to enhance the toxicity of Formula 13. All detoxification enzyme inhibitors significantly increased the toxicity of Formula 13 with PBO being the most capable of producing strong synergism.

FIG. 11 shows dose-response curves of Formula 30 applied alone or in combination with either a solution of 1% PBO or 1% CWT. Both PBO and cedarwood (Texas type) oil (CWT) were capable of shifting the dose-response curve to the left, indicating a synergistic interaction with this pogostone derivative.

FIG. 12 shows the results of soil drench experiments that demonstrate the herbicidal properties of the disclosed pogostone derivatives. Foxtail and dandelions were exposed to 40 mL of 0.5% Formula 13 or 2,4-dichlorophenoxyacetic acid (2,4-D). Formula 13 caused significant phytotoxicity in the foxtail spp., whereas little phytotoxicity was noted in the dandelion exposure. This trend (i.e., more toxic to foxtail and less toxic to dandelions) was reversed in the 2,4-D exposure group.

FIG. 13 shows an exemplary synthesis scheme for preparing pogostone with dehydroacetic acid (DHAA).

### DETAILED DESCRIPTION

Described herein are novel pogostone derivatives and methods of using the same as insecticides, repellents, larvicides, fungicides, anti-microbials, antibiotics, and/or herbicides.

Resistance to numerous insecticides has developed in wild pest arthropod populations. For example, numerous populations of Aedes aegypti in southern North America have also shown to be resistant to a variety of insecticides representing a significant public health crisis. Moreover, numerous pests have been characterized as resistant to at least one insecticide in approximately 600 independent species of pest arthropod according to at least one study from 2010 (see, e.g., pesticideresistance.org). Because of the paucity of available chemical control agents, new chemical agents need to be developed and utilized for the control of arthropods populations, which can limit food security and vector significant veterinary and human disease agents. The recent Zika virus epidemic that has spread throughout South and Central America and the Caribbean has further demonstrated arthropod- or insect-borne disease is a significant threat to mankind, and the development of novel control technologies must be a paramount objective for our global society.

So far in various insect bioassays the disclosed pogostone derivatives performed similarly, if not better, than natural pyrethrins at killing and/or knocking down numerous pest insects and arthropods. As discussed in more detail below, the disclosed pogostone derivatives have been assessed in numerous assays and against numerous arthropod pests relevant to a variety of pest control markets (e.g., cockroaches, house flies, mosquitoes, European corn borer, western corn rootworm, and stored product pests). The novel pogostone derivatives disclosed herein represent a new class of chemistry that could be utilized to combat pest arthropods. Due to their lack of structural similarity to any currently available insecticide on the market, it is likely that they possess a unique mechanism of action compared to other insecticides. Moreover, the symptomology associated with intoxication by these compounds is distinct and quite promising. Rapid immobilization is observed after the application of these compounds to target arthropods, followed by subsequent mortality within 24-hr. The speed at which these compounds immobilize exposed arthropods rivals or bests currently available pyrethroids, making the disclosed compounds well-suited for inclusion in insecticidal and/or arthropod repellent formulations, and additional data suggests that the disclosed compounds may be used as larvicides, antibiotics, anti-microbials, and/or herbicides as well.

### I. Definitions

As used in the description of the invention and the appended claims, the singular forms “a”, “an” and “the” are used interchangeably and intended to include the plural forms as well and fall within each meaning, unless the context clearly indicates otherwise. Also, as used herein, “and/or” refers to and encompasses any and all possible combinations of one or more of the listed items, as well as the lack of combinations when interpreted in the alternative (“or”).

As used herein, the term “about” will be understood by persons of ordinary skill in the art and will vary to some extent depending upon the context in which it is used. If there are uses of the term which are not clear to persons of ordinary skill in the art given the context in which it is used, “about” will mean up to plus or minus 10% of the particular term.

As used herein, the phrase “effective amount” means a dosage or amount that provides the specific effect for which the compound is administered, i.e., to reduce, kill, repel, or otherwise eliminate undesirable organisms such as, for example, insects, insect larva, fungi, and weeds. It is emphasized that an effective amount may not always be effective in reducing, killing, repelling, or eliminating the undesirable organisms described herein, even though such dosage is deemed to be generally effective by those of skill in the art. Further, an effective amount may vary based on the way/form in which the disclosed pogostone derivatives are applied, the undesirable organism being killed/repelled/eliminated, and/or whether the disclosed pogostone derivative is applied alone or in combination with one or more other compounds, including but not limited to baits.

As used herein, the term “insecticide” means a compound that is toxic to insects and, optionally, toxic to other arthropods as well. Similarly, an “insect repellent” may repel organisms belonging to the class Insecta, but may also optionally function to repel other classes of organisms within the phylum of arthropods.

### II. Pogostone

Pogostone, or 3-(4-Methylpentanoyl)-4-hydroxy-2H, is a chemical that was isolated from Pogostemon cablin, and which possesses a variety of potential activities, including but not limited to, insecticidal, larvicidal, antibacterial, anticandida, antifungal, and/or herbicidal activities. Pogostone can also be isolated from patchouli oil. The chemical formula for pogostone is shown in Formula I below

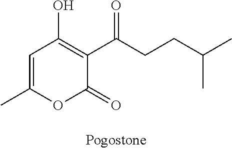 [SMILES: Cc1cc(O)c(C(=O)CCC(C)C)c(=O)o1]

### III. Pogostone Derivatives

The present disclosure provides numerous active derivatives of pogostone and salts thereof. The disclosed compounds represent potential insecticides, larvicides, repellents, and knockdown agents, and may also be used as fungicides, antibiotics, anti-microbials, and/or herbicides. In terms of insecticidal properties, all of the disclosed compounds performed better than terpenoids, which have been utilized and described as insecticides and repellents in a variety of products and patents. In general, the disclosed pogostone derivatives and salts thereof usually comprise the same overall core structure:

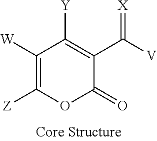

wherein V may be a substituted or unsubstituted C₃-C₁₂ unbranched or branched alkyl, substituted or unsubstituted C₂-C₁₂ unbranched or branched alkenyl, substituted or unsubstituted C₃-C₁₂ unbranched or branched alkynyl, substituted or unsubstituted C₃-C₁₂ unbranched or branched cycloalkyl, substituted or unsubstituted heteroaryl, and substituted or unsubstituted C₃-C₁₂ unbranched or branched cycloalkenyl;
wherein W may be a hydrogen or a substituted or unsubstituted C₃-C₆ saturated or unsaturated alkane or alkene;
wherein X may be an oxygen or sulfur atom;
wherein Y may be a hydroxyl group, sulphhydryl, amino, a halogen, or an ether with variable alkyl length (C₁-C₇);
wherein Z may be a substituted or unsubstituted C₁-C₁₂ unbranched or branched alkyl, substituted or unsubstituted C₃-C₁₂ unbranched or branched cycloalkyl, substituted or unsubstituted heteroaryl, and substituted or unsubstituted C₃-C₁₂ unbranched or branched cycloalkenyl; and
wherein W may be linked to Z via a substituted or unsubstituted C₃-C₆ saturated or unsaturated alkane or alkene.

More specifically, the disclosed derivatives and salts thereof can be divided into to one of three sub-classes that comprise one of the core structures shown in Formulas 1-3 below.

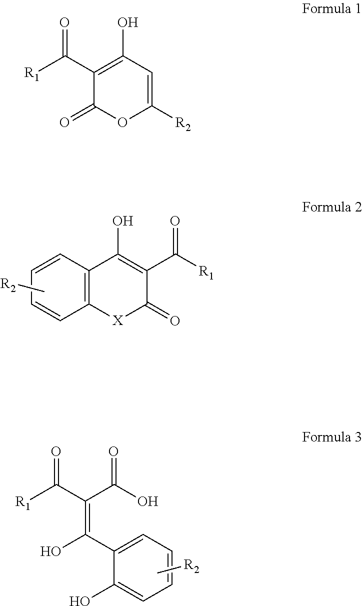

The substituent groups of R₁ may be a substituted or unsubstituted C₁-C₁₂ unbranched or branched alkyl, substituted or unsubstituted C₂-C₁₂ unbranched or branched alkenyl, substituted or unsubstituted C₃-C₁₂ unbranched or branched alkynyl, substituted or unsubstituted C₃-C₁₂ unbranched or branched cycloalkyl, substituted or unsubstituted heteroaryl, and substituted or unsubstituted C₃-C₁₂ unbranched or branched cycloalkenyl.

The substituent groups of R₂ may be a hydrogen, a substituted or unsubstituted C₃-C₁₂ unbranched or branched alkyl, substituted or unsubstituted C₂-C₁₂ unbranched or branched alkenyl, substituted or unsubstituted C₃-C₁₂ unbranched or branched alkynyl, substituted or unsubstituted C₃-C₁₂ unbranched or branched cycloalkyl, substituted or unsubstituted heteroaryl, and substituted or unsubstituted C₃-C₁₂ unbranched or branched cycloalkenyl, a hydroxyl group, sulphhydryl, amino, a halogen, or an ether with variable alkyl length (C₁-C₇).

The substituent groups of X may be an oxygen, a carbon, or a nitrogen.

As shown in the Examples section below, the disclosed derivatives and salts thereof are potent insecticides and repellents that can rapidly kill and/or knock down a variety of arthropods and insects. Moreover, many of the compounds possess fungicidal, antibacterial, anti-microbial, and herbicidal properties as well. In particular, the disclosed pogostone derivatives are capable of killing or repelling (i.e., “exhibiting a controlling effect on”) arthropods including, but not limited to, those belonging to the classes of Insecta, Acarina, Chilognatha, Epimorpha and Isopoda.

Exemplary compounds that fall generally within each sub-class of Formulas 1-3 are provided herein (some derivatives may have variations to the core formulas, e.g., Formula 15 has a nitrogen (N) in place of oxygen (O) in a heterocyclic ring). For example, exemplary compounds with the common core structure of Formula 1 include:

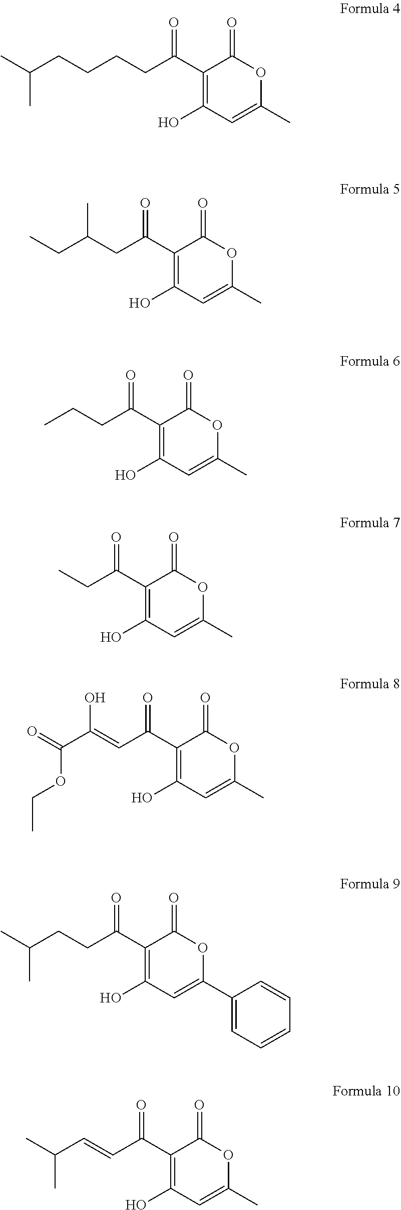

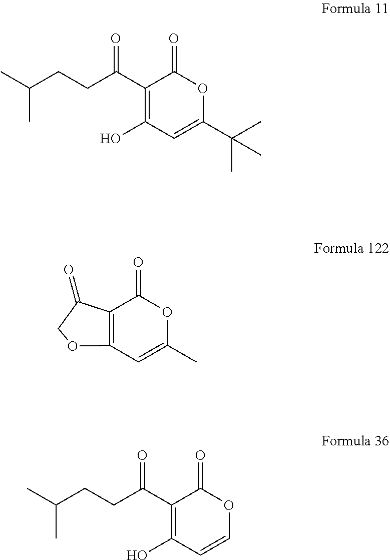

Exemplary compounds that possess the same general common core structure of Formula 2 (slight deviations from the core structure may occur) include:

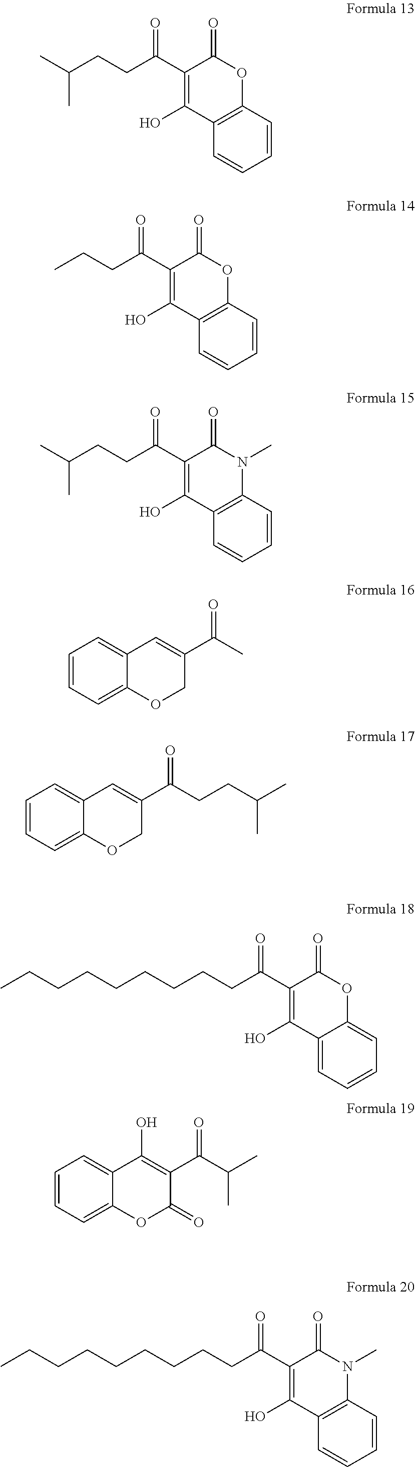

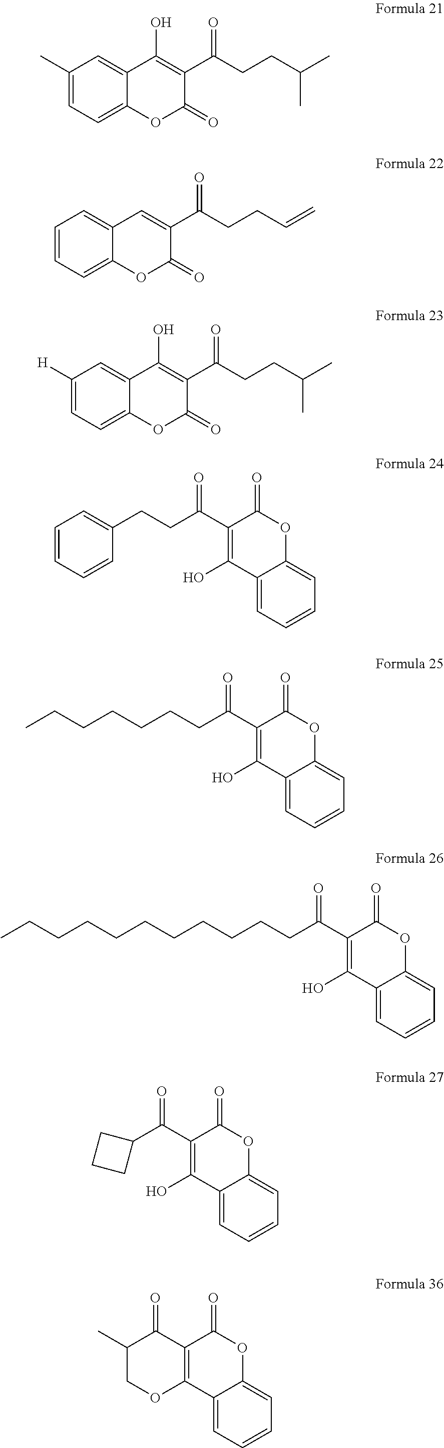

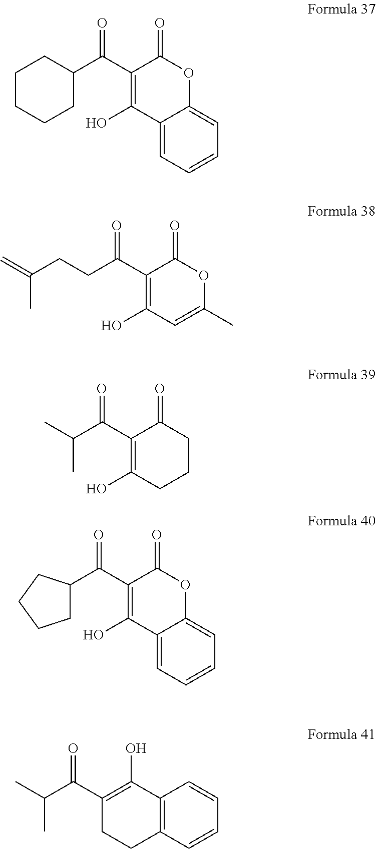

Additional compounds that possess the same general common core structure of Formula 2 (slight deviations from the core structure may occur) include:

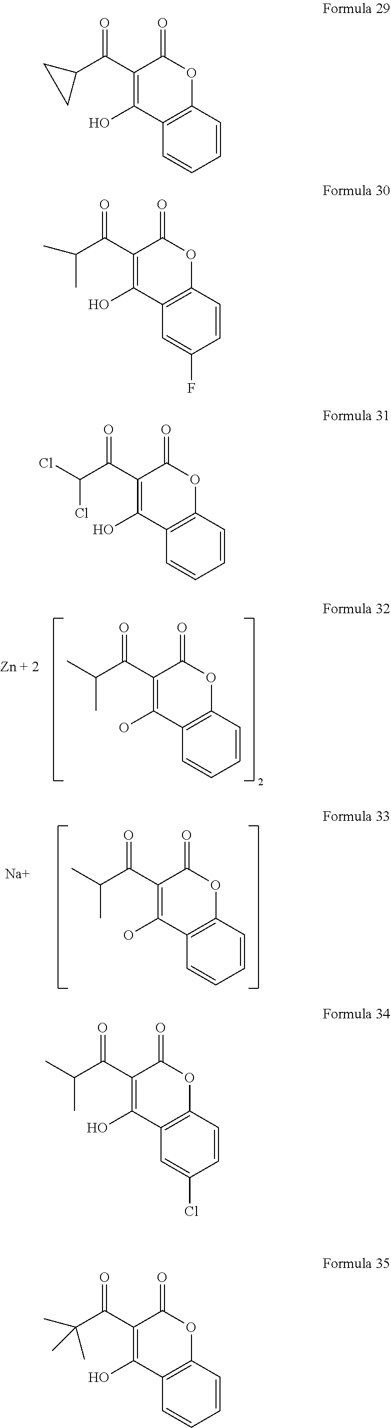

Exemplary compounds with the common core structure of Formula 3 (Formula 28 generally corresponds to the structure of Formula 3, but has had the hydroxyl group at position 5 of the benzene ring removed) include:

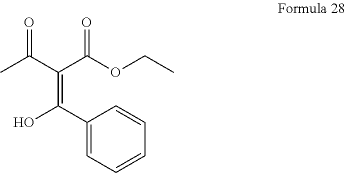 [SMILES: CCOC(=O)/C(C(C)=O)=C(/O)c1ccccc1]

Other suitable substituent groups that may be incorporated into the R₁ and R₂ positions of Formulas 1-3 include, but are not limited to, a hydrogen atom, a halogen atom, a hydroxy group, a formyl group, a C₁-C₁₂ alkyl group, a C₁-C₁₂ alkoxy group, a C₃-C₆ cycloalkyl group, a C₁-C₁₂ alkylcarbonyl group, a C₁-C₁₂ alkylsulfonyl group, a di-C₁-C₈ alkylamino-C₁-C₈ alkyl group, a C₂-C₁₂ alkenyl group, a C₂-C₁₂ alkynyl group, a C₃-C₆ cycloalkenyl group, a bicyclo-C₅-C₈ cycloalkyl group, a phenyl group, or a 5- or 6-membered aromatic heterocyclic group.

For the purposes of the present disclosure, a “C₁-C₁₂ alkyl group” refers to a linear or branched alkyl group having 1 to 12 carbon atoms. Non-limiting examples thereof include a methyl group, an ethyl group, a propyl group, an isopropyl group, a butyl group, an isobutyl group, a sec-butyl group, a tert-butyl group, a pentyl group, an isopentyl group, a 2-methylbutyl group, a neopentyl group, a 1-ethylpropyl group, a hexyl group, an isohexyl group, and a 4-methylpentyl group.

For the purposes of the present disclosure, a “C₃-C₆ cycloalkyl group” may be, but is not limited to, a cyclopropyl group, a cyclobutyl group, a cyclopentyl group, or a cyclohexyl group.

For the purposes of the present disclosure, a “C₁-C₁₂ alkoxy group” refers to a C₁-C₁₂ alkoxy group formed from the C₁-C₁₂ alkyl group mentioned above. Non-limiting examples thereof include a methoxy group, an ethoxy group, an n-propoxy group, an isopropoxy group, a butoxy group, an isobutoxy group, a sec-butoxy group, a tert-butoxy group, a pentoxy group, an isopentoxy group, a 2-methylbutoxy group, a hexyloxy, and an isohexyloxy group.

For the purposes of the present disclosure, a “C₁-C₁₂ alkylcarbonyl group” refers to a group in which a carbonyl group is substituted by one C₁-C₁₂ alkyl group mentioned above. Non-limiting examples thereof include an acetyl group, an ethylcarbonyl group, a propylcarbonyl group, and an isopropylcarbonyl group.

For the purposes of the present disclosure, a “C₁-C₁₂ alkylene group” refers to a group in which one C₁-C₁₂ alkyl group mentioned above forms a divalent substituent. Non-limiting examples thereof include a methylene group, an ethylene group, a propylene group, and a butylene group.

For the purposes of the present disclosure, an “oxy-C₁-C₆ alkylene group” refers to a group in which one C₁-C₁₂ alkylene group mentioned above is substituted by one oxy group. Non-limiting examples thereof include an oxymethylene group and an oxyethylene group.

For the purposes of the present disclosure, a “C₂-C₁₂ alkenyl group” refers to a linear or branched alkenyl group having 2 to 12 carbon atoms. Non-limiting examples thereof include a vinyl group, an allyl group, a 1-propenyl group, an isopropenyl group, a 2-methyl-1-propenyl group, a 2-methyl-2-propenyl group, a 1-butenyl group, a 2-butenyl group, a 3-butenyl group, a 1-pentenyl group, a 1-hexenyl group, a 1,3-hexadienyl group, and a 1,5-hexadienyl group.

For the purposes of the present disclosure, a “C₂-C₁₂ alkynyl group” refers to a linear or branched alkynyl group having 2 to 12 carbon atoms. Non-limiting examples thereof include an ethynyl group, a 1-propynyl group, a 2-propynyl group, a 1-butynyl group, a 2-butynyl group, a 3-butynyl group, a 1-ethynyl-2-propynyl group, a 1-methyl-2-propynyl group, a 1-pentynyl group, a 1-hexynyl group, a 1,3-hexadiynyl group, and a 1,5-hexadiynyl group.

For the purposes of the present disclosure, a “C₁-C₆ alkoxy-C₁-C₆ alkyl group” refers to a group in which the C₁-C₁₂ alkyl group mentioned above is substituted by one C₁-C₆ alkoxy group mentioned above. Non-limiting examples thereof include a methoxymethyl group, an ethoxymethyl group, a propoxymethyl group, an isopropoxymethyl group, a methoxyethyl group, an ethoxyethyl group, a propoxyethyl group, and an isopropoxyethyl group.

For the purposes of the present disclosure, a “C₁-C₁₂ alkylsulfonyl group” refers to a group in which a sulfonyl group is substituted by one C₁-C₆ alkyl group mentioned above. Non-limiting examples thereof include a methylsulfonyl group, an ethylsulfonyl group, a propylsulfonyl group, and an isopropylsulfonyl group.

For the purposes of the present disclosure, a “di-C₁-C₆ alkylamino-C₁-C₆ alkyl group” refers to a group in which the C₁-C₆ alkyl group mentioned above is substituted by an amino group substituted by two C₁-C₆ alkyl groups mentioned above. Non-limiting examples thereof include a dimethylaminomethyl group, a dimethylaminoethyl group, and a dimethylaminopropyl group.

For the purposes of the present disclosure, a “C₃-C₆ cycloalkenyl group” may be a cyclopropenyl group, a cyclobutenyl group, a cyclopentenyl group, or a cyclohexenyl group.

The disclosed pogostone derivatives may be prepared and used as individual enantiomers or racemic or diestereomer mixtures.

Additional derivatives that can be derived from the common core structures of formulas 1, 2, or 3 include, but are not limited to:

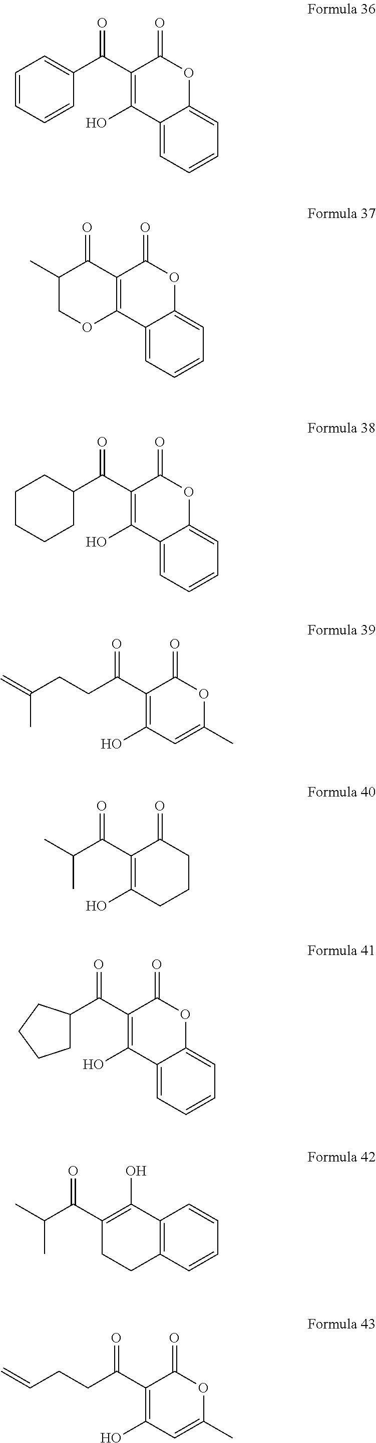

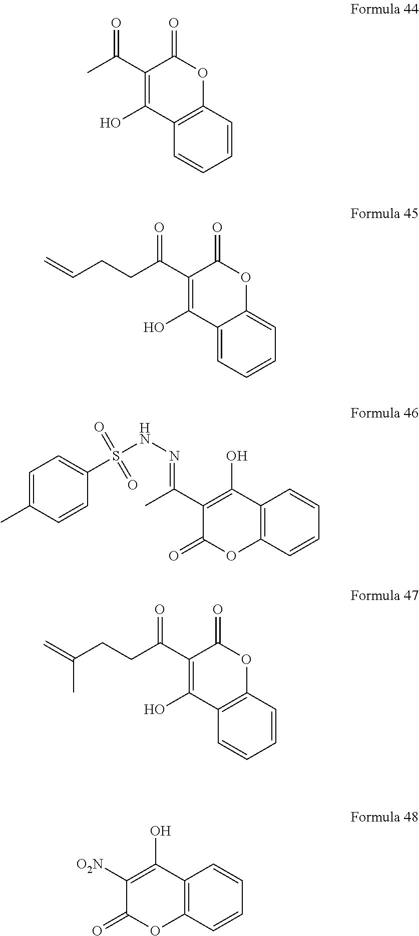 [SMILES: CC(=O)c1c(O)c2ccccc2oc1=O ; C=CCCC(=O)c1c(O)c2ccccc2oc1=O ; C/C(=N\NS(=O)(=O)c1ccc(C)cc1)c1c(O)c2ccccc2oc1=O ; C=C(C)CCC(=O)c1c(O)c2ccccc2oc1=O ; O=c1oc2ccccc2c(O)c1[N+](=O)[O-]]

In addition to the foregoing derivatives, the present disclosure provides pogostone derivatives that can be prepared using a dehydroacetic acid (DHAA)-based synthesis method (detailed in Section IV below). For the purposes of the present disclosure, the derivatives synthesized using a DHAA-based scheme are collectively referred to as DHAA-based derivatives.

The DHAA-based derivatives of the present disclosure include, but are not limited to:

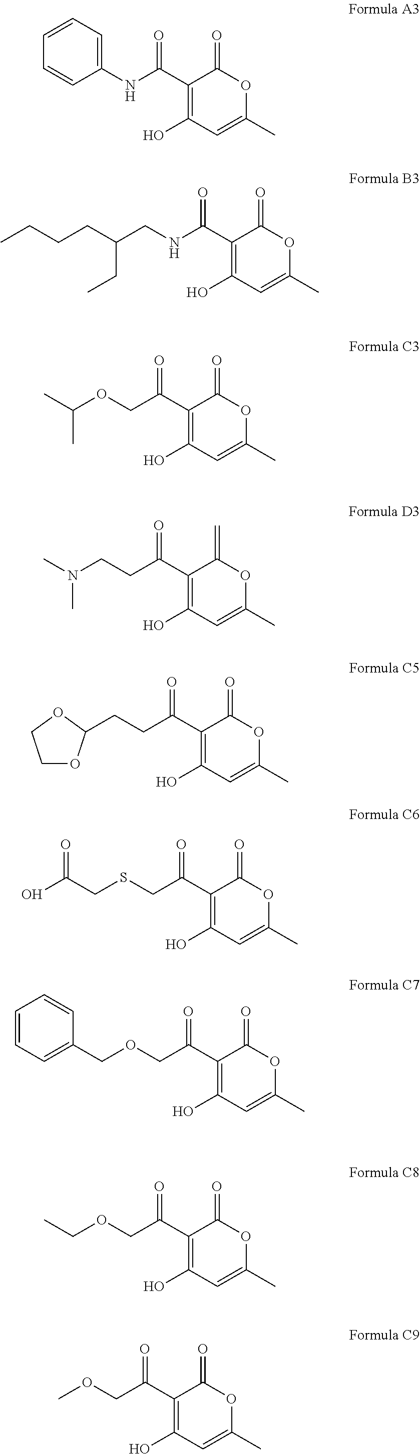

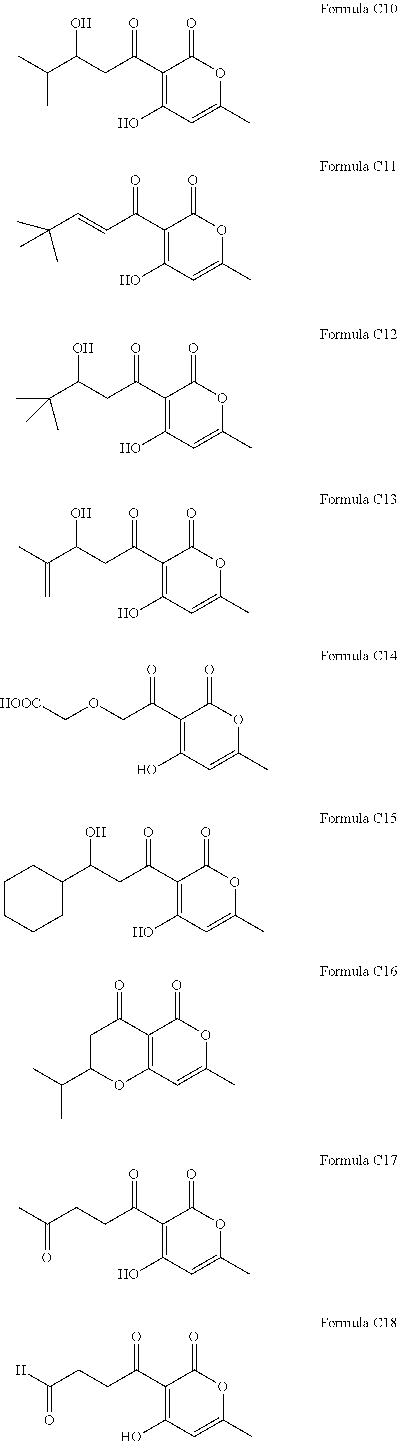

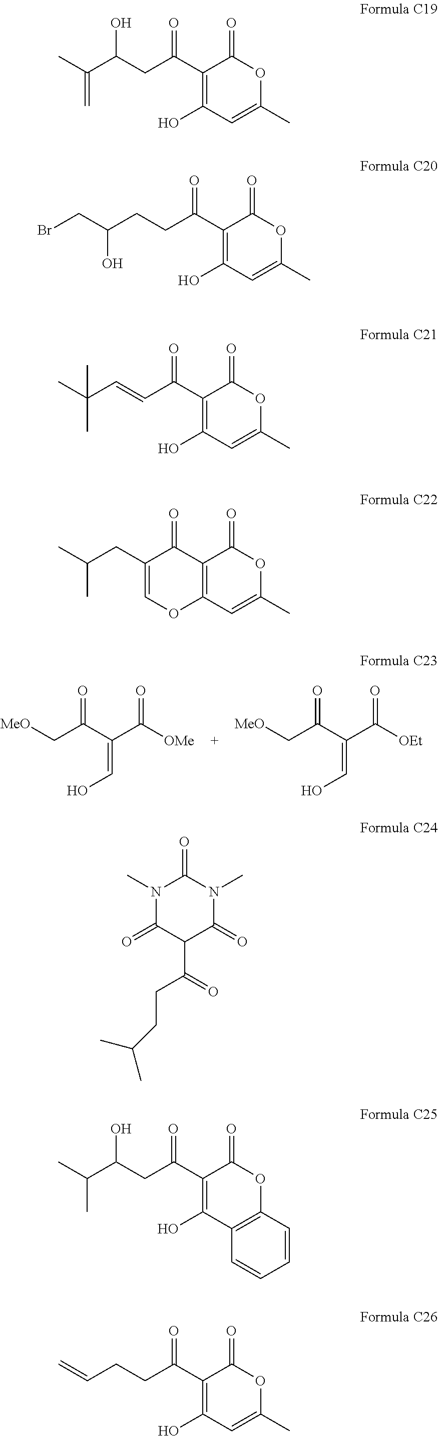

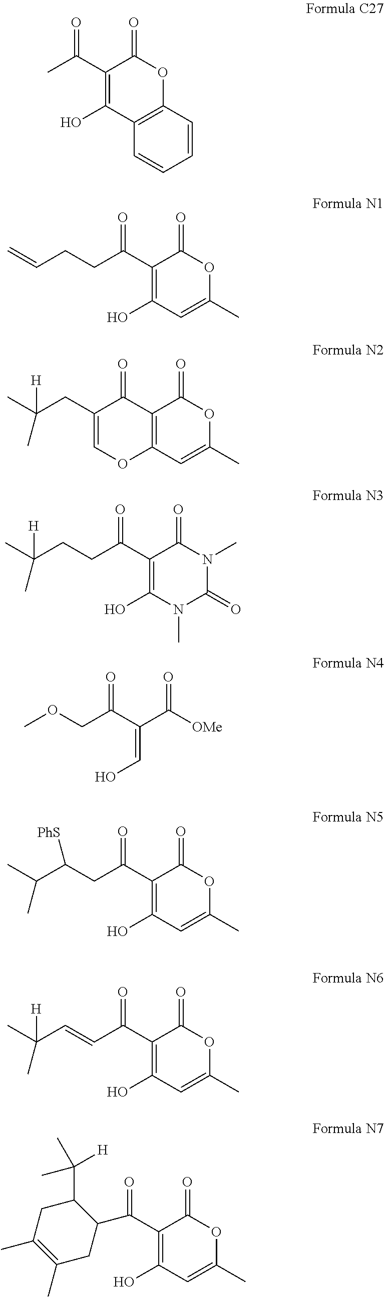

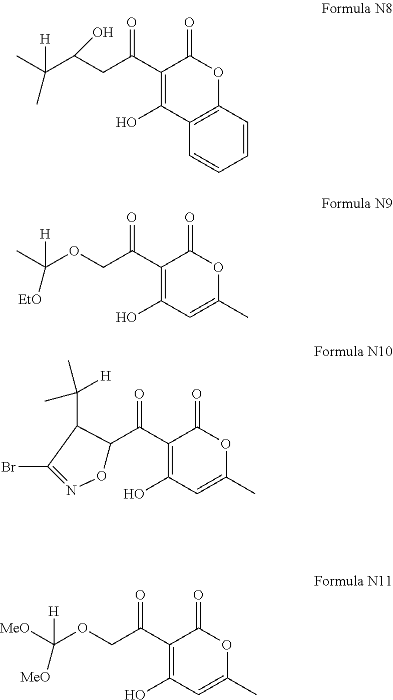 [SMILES: CC(C)C(O)CC(=O)c1c(O)c2ccccc2oc1=O ; CCOC(C)OCC(=O)c1c(O)cc(C)oc1=O ; Cc1cc(O)c(C(=O)C2ON=C(Br)C2C(C)C)c(=O)o1 ; COC(OC)OCC(=O)c1c(O)cc(C)oc1=O]

The foregoing DHAA-based derivatives provide the commercial benefit of not requiring triacetic acid lactone (TAL), which may be used for the synthesis of some embodiments of the disclosed pogostone derivatives.

### IV. Methods of Making the Disclosed Derivatives

Exemplary methods of making the disclosed derivatives and salts thereof are included herein. For instance, exemplary methods for making the compound of Formula 6 are disclosed below.

First, synthesis may begin using the commercially available 1. Using 1 and commercially available 4-methylpentanoic acid 4 as the starting material a reaction can be conducted. N,N′-Dicyclohexylcarbodiimide (DCC) and 4-Dimethylaminopyridine (DMAP) are known as synthetically useful and mild reagents for the preparation of esters and amides. So using these reagents, a person of ordinary skill in the art would be able to get a one-step preparation of 3-acyl-4-hydroxy-2-pyrone from carboxylic acids and 1. This reaction goes through a simultaneous Fries type rearrangement of O-enol acyl group of 1 towards a position of the lactone to get the desired C-acylation product as illustrated in Scheme 1.

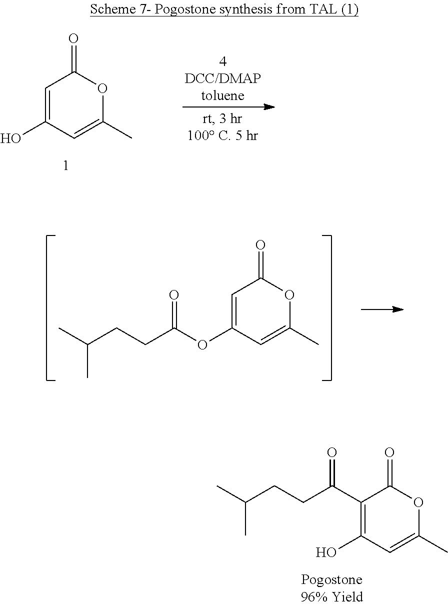

The reaction can be carried out under argon at room temperature for 3 hours heating to 100° C. for 5 hours. Subjecting the crude material to flash column chromatography for purification afford the derivative in approximately 96% yield, which the best yield for pyrone that has ever been reported. The reaction was first carried out in 2.0 mmol scale, but later this one step reaction was scaled up to 10 grams and gave similar results. Next, we used the bio-based TAL as the starting material which we obtained from CBiRC collaboration. There were two bio-based samples which were differing from their purification methods from the biomass broth as 100% and 94% purity. When these were subjected to the one step synthesis of 3, 99% and 93% yields were achieved for the 100% pure TAL and 94% pure TAL, respectively.

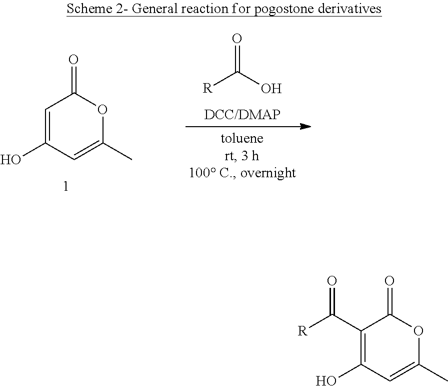

In order to get different analogs of pogostone, the same reaction procedure as in Scheme 2 can be followed using the relevant acids. Here, depending on the acids, the heating time for the reaction may be changed from 5 hours to, e.g., overnight at 100° C.

Pogostone can also be derived, for the purposes of preparing the disclosed derivatives by flaming dried flask of commercially available 98% purity TAL (0.252 g, 2.0 mmol). Dried toluene (5 mL) can be added to this flask and stirred. To this suspension, DCC (0.413 g, 2.0 mmol) and DMAP (0.048 g, 0.4 mmol) may be added and stirred under argon. Then 4-methylpentanoic acid (0.25 mL, 2.0 mmol) may be added to this mixture and was stirred for 3 h at room temperature and was further stirred for 5 h at 100° C. After cooling to room temperature, the reaction mixture can be subjected to filtration. The filtrate can be washed twice with toluene (10 mL×2). The combined toluene solution can then be concentrated under reduced pressure to give the crude product, which was subjected to silica column chromatography (silica gel, hexane:EtOAc 10:1 as an eluent) to afford pogostone (0.429 g, 96%) as a yellow solid.

Further exemplary methods of preparing pogostone derivatives can employ dehydroacetic acid (DHAA), which avoids the need for TAL, DCC, DMAP, and 4-methylpentanoic acid. For example, under a nitrogen atmosphere, dehydroacetic acid (e.g., 10.0 g), isobutyraldehyde (e.g., 6.3 g) and 4A MS powder (e.g., 10.0 g) can be added to dry tetrahydrofuran (e.g., 60 mL). A solution of piperidine (e.g., 2.0 g) in dry tetrahydrofuran (e.g., 15 mL) can then be added dropwise slowly over about 1.5 hours at room temperature while stirring. After the completion of dropwise addition, the solution can be stirred for about 0.5 hours at room temperature. Next, the solution can be filtered, for example via Celite, and washed with ethyl acetate (e.g., 75 mL). The filtrate can be concentrated and the residue can be diluted with ethyl acetate (e.g., 150 mL), washed with hydrochloric acid (e.g., 1N, 100 mL), water (e.g., 100 mL) and brine (e.g., 100 mL). The organic layer can be dried with anhydrous sodium sulfate and concentrated. The product can then be purified using silica gel column chromatography (e.g., 7.0 g, 54%). Synthesis scheme 3 below

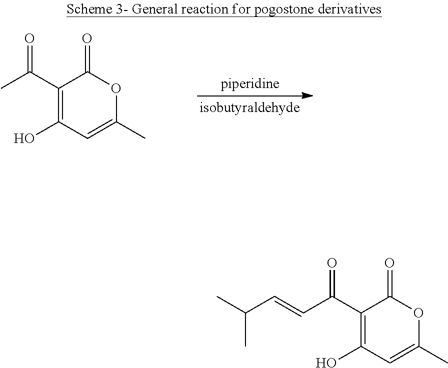 [SMILES: CC(=O)c1c(O)cc(C)oc1=O ; Cc1cc(O)c(C(=O)/C=C/C(C)C)c(=O)o1]

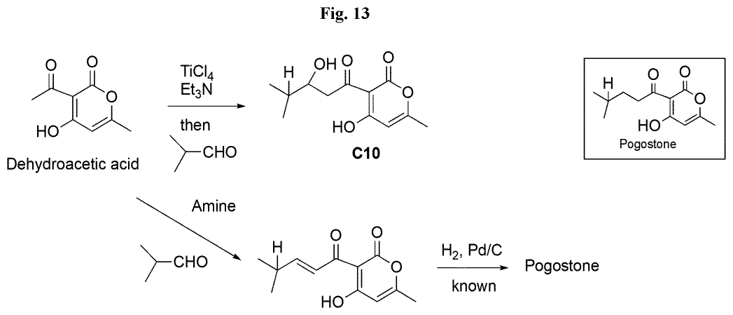

A more detailed version of this scheme is shown in FIG. 13. This DHAA-based chemistry can be used to prepare the C-series derivatives described above.

### V. Methods of Using the Disclosed Derivative

The disclosed pogostone derivatives and salts thereof may be used as insecticides, larvicides, fungicides, antibiotics, anti-microbials, herbicides, or arthropod repellents, either alone or in combination with one or more additional pogostone derivatives and salts thereof or known compounds that possess the desirable function (e.g., known insecticides or repellents).

In some embodiments, the disclosed pogostone derivatives and salts thereof may be applied to a plant, part of a plant (e.g., the leaves), plant seeds, or an animal for the purposes of serving as an insecticide, fungicide, larvicide, antibiotic, anti-microbial, herbicide, or arthropod repellent.

In embodiments in which the disclosed pogostone derivatives and salts thereof are used as an insecticide, larvicide, or insect repellent, the target arthropod may be, but is not limited to, delphacidae (planthoppers) such as Laodelphax striatellus (small brown planthopper), Nilaparvata lugens (brown planthopper) and Sogatella furcifera (white-backed rice planthopper); Deltocephalidae (leafhoppers) such as Nephotettix cincticeps (green rice leafhopper), Recilia dorsalis (zig-zag rice leaf hopper) and Nephotettix virescens (green rice leafhopper), Aphididae (aphids) such as cotton aphid (Aphis gossypii); stink bugs; Aleyrodidae (whiteflies) such as Bemisia argentifolii; scales; Tingidae (lace bugs); Psyllidae (suckers); Pyralidae such as Chilo suppressalis (rice stem borer), Cnaphalocrocis medinalis (rice leafroller) and Plodia interpunctella (Indian meal moth); Noctuidae such as Spodoptera litura (tobacco cutworm), Pseudaletia separata (rice armyworm), Mamestra brassicae (cabbage armyworm), Agrotis spp. (e.g. Agrotis segetum (turnip cutworm), Agrotis ipsilon (black cutworm)), Helicoverpa spp., Heliothis spp. and Plusiinae; Pieridae such as Pieris rapae crucivora (common cabbageworm); Tortricidae such as Adoxophyes spp. (e.g. Adoxophyes orana fasciata); Carposinidae such as Carposina niponensis (peach fruit moth); Lyonetiidae; Lymantriidae; Plutellidae such as Plutella xylostella (diamondback moth); Hesperiidae such as Parnara guttata (rice skipper); Tineidae such as Tinea pellionella (casemaking clothes moth) and Tineola bisselliella (webbing clothes moth); Culicidae (mosquitoes) such as Culex spp. (e.g. Culex pipiens pallens (common mosquito), Culex tritaeniorhynchus), Aedes spp. (e.g. Aedes aegypti (yellow fever mosquito), Aedes alhopictus) and Anopheles spp. (e.g. Anopheles sinensis); Chironomidae (midges); Muscidae such as Musca domestica (housefly), Muscina stabulans (false stablefly) and Fannia canicularis (little housefly); Calliphoridae; Sarcophagidae; Anthomyiidae such as Delia platura (seedcorn maggot) and Delia antiqua (onion maggot); Tephritidae (fluit flies); Drosophilidae; Psychodidae (moth flies); Tabanidae; Simuhidae (black flies); Stomoxyidae; Phoridae; Ceratopogonidae (biting midges); Scarabaeidae (scarabs) such as Anomala cuprea (cupreous chafer) and Anomala rufocuprea (soybean beetle); Curculionidae (weevils) such as Sitophilus zeamais (maize weevil), Lissorhoptrus oryzophilus (ricewater weevil), ball weevil and Callosobruchus chinensis (adzuki bean weevil); Dermestidae such as Authrenus verbasci (varied carpet beetle) and Attagenus unicolor japonicus (black carpet beetle); Tenebrionidae (darkling beetles) such as Tenebrio molitor (yellow mealworm) and Triboium castaneum (red flour beetle); Chrysomelidae (leaf beetles) such as Oulema oryzae (rice leaf beetle), Phyllotreta striolata (striped flea beetle) and Aulacophora femoralis (cucurbit leaf beetle); Corn rootworms such as Diabrotica virgifera (western corn rootworm) and Diabrotica undecimpunctata howardi (southern corn rootworm); Anobiidae; Coccinellidae (ladybirds) such as Epilachna spp. (e.g. Epilachna vigintioctopunctata (twenty-eight-spotted ladybird)); Lyctidae (powderpost beetles); Bostrychidae (false powderpost beetles); Cerambycidae; Staphylinidae such as Paederus fuscipes (robe beetle); Blattella germanica (German cockroach); Periplaneta fuliginosa (smokybrown cockroach); Periplaneta americana (American cockroach); Periplaneta brunnea (brown cockroach); Blatta orientalis (oriental cockroach); Thrips palmi, western flower thrips, Thrips hawaiiensis (flower thrips); Formicidae (ants) such as Formica japonica, field ant (Lasius fuliginosus), little red ant (Monomorium pharaonis), little ant (Monomorium nipponensis) and pavement ant (Teramorium caespitum); Vespidae (hornets); Polistes spp. (long-legged wasps); Bethylidae; Tenthredinidae (sawflies) such as Athalis rosae ruficornis (cabbage sawfly); Gryllotalpidae (mole crickets); Acrididae (grasshoppers); Siphonaptera Pests (Fleas); Ctenocephalides canis (dog flea); Ctenocephalides felis (cat flea); Pulex irritans; Pediculus corporis; Pediculus humanus (body louse); Pthirus pubis (crab louse); Reticulitermes speratus; Coptotermes formosanus; Tetranychus cinnabarinus (carmine spider mite); Tetranychus urticae (two-spotted spider mite); Tetranychus kanzawai (Kanzawa spider mite); Panonychus citri (citrus red mite); Panonychus ulmi (European red mite); Boophilus microplus; Haemaphysalis longiconis; House-Dust Mites; Acaridae such as Tyrophagus putrescentiae (copra mite) and Aleuroglyphus ovatus (brown legged grain mite); Dermanyssidae such as Dermatophagoides farinae (American house dust mite) and Dermatophagoides pteronyssinus; Glycyphagidae such as Glycyphagus privatus, Glycyphagus domesticus and Glycyphagus destructor; Cheyletidae such as Chelacaropsis malaccensis and Cheyletus fortis; Tarsonemidae; Chortoglyphus spp.; Haplochthonius spp.; Diplopoda (Milpedes); Chilognatha such as Oxydus spp.; Chilopoda (Centipedes); Scolopendra suhspinipes mutilans, red centipede; Oniscoidea (pill bugs) such as Porcellio spp. (e.g. Porcellio scaber), Porcellionides spp. and Armadillidium spp. (e.g. Armadillium vulgare) and so on. In particular embodiments, the target arthropod that is repelled or killed is selected from the group consisting of blood-sucking insects, biting insects, cockroaches, mosquitoes, blackfly, fleas, house flies, barn fly, face fly, bush fly, deer fly, horse fly, gnats, beetle, beer bug, louse, bed bug, earwig, ant, aphid, spruce bud worm, corn borer, sand flea, tsetse fly, assassin bug, biting flies, sand fly, stored grain pests, clothes moths, ticks, mites, spiders, phytophagous pests, and hematophagous pests. Accordingly, disclosed herein are methods of killing and/or repelling numerous types and species of arthropods.

In embodiments in which the disclosed pogostone derivatives are and salts thereof used as a fungicide, the target fungus may be, but is not limited to, Blast (Pyricularia oryzae), Helminthosporium leaf spot (Cochliobolus miyabeanus), Sheath blight (Rhizoctonia solani), “Bakanae” disease (Gibberella fujikuroi), Wheat Powdery mildew (Erysiphe graminis f. sp. hordei; f sp. tritici), Leaf stripe (Pyrenophora graminea), Net blotch (Pyrenophora teres), Fusarium blight (Gibberella zeae), Stripe rust (Puccinia striiformis, Stem rust (P. graminis), Brown rust (P. recondita), Brown rust (P. hordei), Snow rot (Typhula sp.; Micronectriella nivalis), Loose smut (Ustilago tritici; U. nuda), Eye spot (Pseudocercosporella herpotrichoides), Rhynchosporium leaf blotch (Rhynchosporium secalis), Septoria leaf blotch (Septoria tritici), Glume blotch (Leptosphaeria nodorum), Grape Powdery mildew (Uncinula necator), Anthracnose (Elsinoe ampelina), Ripe rot (Glomerella cingulata), Rust (Phakopsora ampelopsidis), Apple Powdery mildew (Podosphaera leucotricha), Scab (Venturia inaequalis), Alternaria leaf spot (Alternaria mali), Rust (Gymnosporangium yamadae), Blossom blight (Sclerotinia mali), Canker (Valsa mali), Pear Black spot (Alternaria kikuchiana), Scab (Venturia nashicola), Rust (Gymnosporangium haraeanum), Peach Brown rot (Sclerotinia cinerea), Scab (Cladosporium carpophilum), Phomopsis rot (Phomopsis sp.), Persimmon Anthracnose (Gloeosporium kaki), Angular leaf spot (Cercospora kaki; Mycosphaerella nawae), Melon Powdery mildew (Sphaerotheca fuliginea), Anthracnose (Colletotri chum lagenarium), Gummy stem blight (Mycosphaerella melonis), Tomato Early blight (Alternaria solani), leaf mold (Cladosporium fulvam), Eggplant Powdery mildew (Erysiphe cichoracoarum), Alternaria leaf spot (Alternaria japonica), White spot (Cerocosporella barassicae), Leak Rust (Puccinia allii), Beans Purple spec (Cercospora kikuchii), Sphaceloma scab (Elsinoe glycines), Pod and stem blight (Diaporthe phaseololum), Beans Anthracnose (Colletotrichum lindemuthianum), Leaf spot (Mycosphaerella personatum), Brown leaf spot (Cercospora arachidicola), Powdery mildew (Erysiphe pisi), Early blight (Alternaria solani), Net blister blight (Exobasidium reticulatum), White scab (Elsinoe leucospila), Brown spot (Alternaria longipes), Beans Powdery mildew (Erysiphe cichoracearum), Anthracnose (Colletotrichum tabacum), Cercospora leaf spot (Cercospora beticola), Black spot (Diplocarpon rosae), Powdery mildew (Sphaerotheca pannosa), Leaf blotch (Septoria chrysanthemi-indici), Rust (Puccinia horiana), Powdery mildew (Sphaerotheca humuli), Gray mold (Botrytis cinerea), and Sclerotinia rot (Sclerotinia sclerotiorum).

In embodiments in which the disclosed pogostone derivatives and salts thereof are used as an herbicide, the target plant may include, but is not limited to, grasses, broadleaf weeds, sedge weeds, and combinations thereof. In some embodiments, the compositions disclosed herein can be used for controlling undesirable vegetation including, but not limited to, Polygonum spp. such as wild buckwheat (Polygonum convolvulus), Amaranthus spp. such as pigweed (Amaranthus retroflexus), Chenopodium spp. such as common lambsquarters (Chenopodium album L.), Sida spp. such as prickly sida (Sida spinosa L.), Ambrosia spp. such as common ragweed (Ambrosia artemisiifolia), Cyperus spp. such as nutsedge (Cyperus esculentus), Setaria spp. such as giant foxtail (Setaria faberi), Sorghum spp., Acanthospermum spp., Anthemis spp., Atriplex spp., Brassica spp., Cirsium spp., Convolvulus spp., Conyza spp., such as horseweed (Conyza canadensis), Cassia spp., Commelina spp., Datura spp., Digitaria spp., Echinochloa spp., Euphorbia spp., Geranium spp., Galinsoga spp., Ipomoea spp. such as morning-glory, Lamium spp., Malva spp., Matricaria spp., Persicaria spp., Prosopis spp., Rumex spp., Sisymbrium spp., Solanum spp., Trifolium spp., Xanthium spp., Veronica spp., Viola spp. such as wild pansy (Viola tricolor), common chickweed (Stellaria media), velvetleaf (Abutilon theophrasti), hemp sesbania (Sesbania exaltata Cory), Anoda cristata, Bidens pilosa, Brassica kaber, shepherd's purse (Capsella bursa-pastoris), cornflower (Centaurea cyanus or Cyanus segetum), hempnettle (Galeopsis tetrahit), cleavers (Galium aparine), common sunflower (Helianthus annuus), Desmodium tortuosum, kochia (Kochia scoparia), Medicago arabica, Mercurialis annua, Myosotis arvensis, common poppy (Papaver rhoeas), Raphanus raphanistrum, Russian thistle (Salsola kali), wild mustard (Sinapis arvensis), Sonchus arvensis, Thlaspi arvense, Tagetes minuta, Richardia brasiliensis, Plantago major, Plantago lanceolata, bird's-eye speedwell (Veronica persica) and speedwell. In some embodiments, the undesirable vegetation includes velvetleaf (Abutilon theophrasti), pigweed (Amaranthus retroflexus), rape (Brassica napus), thistle (Cirsium arvense), nutsedge (CYPES, Cyperus esculentus), large crabgrass (Digitaria sanguinalis), barnyardgrass (Echinochloa crus-galli), poinsettia (Euphorbia heterophylla), common sunflower (Helianthus annuus), ivyleaf morningglory (Ipomoea hederacea), ivy-leaved speedwell (Veronica hederifolia), wild pansy (Viola tricolor), or a combination thereof. In certain embodiments, the undesirable vegetation that can be killed or reduced by the disclosed pogostone derivatives includes pigweed (Amaranthus retroflexus), poinsettia (Euphorbia heterophylla), nutsedge (Cyperus esculentus), morning glory (Ipomoea hederacea), or a combination thereof.

Further, in some embodiments, the disclosed pogostone derivatives can be used to control herbicide resistant or tolerant weeds. The methods employing the compositions described herein may also be employed to control herbicide resistant or tolerant weeds. Exemplary resistant or tolerant weeds include, but are not limited to, biotypes resistant or tolerant to acetolactate synthase (ALS) or acetohydroxy acid synthase (AHAS) inhibitors (e.g., imidazolinones, sulfonylureas, pyrimidinylthiobenzoates, triazolopyrimidines, sulfonylaminocarbonyltriazolinones), acetyl CoA carboxylase (ACCase) inhibitors (e.g., aryloxyphenoxypropionates, cyclohexanediones, phenylpyrazolines), synthetic auxins (e.g., benzoic acids, phenoxycarboxylic acids, pyridine carboxylic acids, quinoline carboxylic acids), auxin transport inhibitors (e.g., phthalamates, semicarbazones), photosystem I inhibitors (e.g., bipyridyliums), 5-enolpyruvylshikimate-3-phosphate (EPSP) synthase inhibitors (e.g., glyphosate), glutamine synthetase inhibitors (e.g., glufosinate, bialafos), microtubule assembly inhibitors (e.g., benzamides, benzoic acids, dinitroanilines, phosphoramidates, pyridines), mitosis inhibitors (e.g., carbamates), very long chain fatty acid (VLCFA) inhibitors (e.g., acetamides, chloroacetamides, oxyacetamides, tetrazolinones), fatty acid and lipid synthesis inhibitors (e.g., phosphorodithioates, thiocarbamates, benzofuranes, chlorocarbonic acids), protoporphyrinogen oxidase (PPO) inhibitors (e.g., diphenylethers, N-phenylphthalimides, oxadiazoles, oxazolidinediones, phenylpyrazoles, pyrimidinediones, thiadiazoles, triazolinones), carotenoid biosynthesis inhibitors (e.g., clomazone, amitrole, aclonifen), phytoene desaturase (PDS) inhibitors (e.g., amides, anilidex, furanones, phenoxybutan-amides, pyridiazinones, pyridines), 4-hydroxyphenyl-pyruvate-dioxygenase (HPPD) inhibitors (e.g., callistemones, isoxazoles, pyrazoles, triketones), cellulose biosynthesis inhibitors (e.g., nitriles, benzamides, quinclorac, triazolocarboxamides), herbicides with multiple modes-of-action such as quinclorac, and unclassified herbicides such as arylaminopropionic acids, difenzoquat, endothall, and organoarsenicals. Exemplary resistant or tolerant weeds include, but are not limited to, biotypes with resistance or tolerance to multiple herbicides, biotypes with resistance or tolerance to multiple chemical classes, biotypes with resistance or tolerance to multiple herbicide modes-of-action, and biotypes with multiple resistance or tolerance mechanisms (e.g., target site resistance or metabolic resistance).

In embodiments in which the disclosed pogostone derivatives and salts thereof are used as an antibiotic, the target bacteria may be, but is not limited to, Xanthomonas campestris pv. Campestris, Clavibacter michiganensis pv. Michiganensis, Pseudomonas spp., Erwinia spp., Xanthomonas campestris-various strains, Ralstonia solanacearum, Pseudomonas syringae—various strains, Pseudomonas syringae pv. Pisi, Pseudomonas syringae pv. Tomato, Pseudomonas syringae pv. Syringae, seudomonas syringae pv. maculicola (brassicas), Pseudomonas spp. (lettuce), Rhizomonas suberifaciens (lettuce), P. syringae pv. lachrymans (cucurbits), Pseudomonas corrugata and other bacteria (tomatoes), Xanthomonas campestris pv. phaseoli (beans), Pseudomonas syringae pv. phaseolicola (beans), Erwinia carotovora pv. atroseptica (potatoes).

In embodiments in which the disclosed pogostone derivatives and salts thereof are used as an anti-microbial, the target microbe may be, but is not limited to, Salmonella spp. Campylobacter spp. E. coli (various strains), Listeria spp. Clostridium perfringens, Staphylococcus aureus, Toxoplasma gondii.

For the purposes of the disclosed methods, a pogostone derivative and salts thereof may be applied to a plant or animal for the purposes of serving as an insecticide, larvicide, fungicide, antibiotic, anti-microbial, herbicide, or arthropod repellent three or more times a day, twice a day, or once a day. In some embodiments, the pogostone derivative may be applied once a day, once every other day, three times a week, twice a week, once a week, once every other week, once every three weeks, once a month, once every other month, once every three months, once every four months, once every five months, once every six months, or less frequently. In such embodiments, the pogostone derivative may be applied to a plant or an animal, either sequentially or concurrently, with one or more additional insecticides, larvicides, fungicides, antibiotics, anti-microbials, herbicides, or arthropod repellents.

For the purposes of the disclosed methods, the pogostone derivatives and salts thereof may be applied to the intended plant, seed, or animal in any appropriate form, such as in a spray, aerosol, liquid, gel, powder, or solid form. The pogostone derivatives and salts thereof may be formulated and applied to a plant or animal as solids, liquids, or gases (e.g., using a vapor delivery system).

In some embodiments, the plant to which the disclosed pogostone derivatives and salts thereof are applied may be a crop plant, such as corn, soybeans, wheat, fruits, vegetables, potatoes, legumes, nuts, cotton, etc. In some embodiments, the animal to which the disclosed pogostone derivatives are applied may be a human, a livestock animal (e.g., a cow, a pig, a horse, a goat, a llama, a sheep, a chicken, etc.), or a companion animal (e.g., a dog or a cat).

In some embodiments, the disclosed pogostone derivatives and salts thereof may be combined with other known compounds to produce a synergistic effect, as discussed in more detail below. Accordingly, disclosed herein are methods of killing or repelling arthropods or insects by applying a combination of one or more of the disclosed pogostone derivatives and a known insecticide or insect repellent (e.g., chlorfenapyr, piperonyl butoxide (PBO), S,S,S-tributyl phosphorotrithioate (DEF), N,N-diethyl-m-toluamide (DEET), etc.). Further examples of combinable insecticides and acaricides including, but not limited to, diethyl maleate (DEM), MGK-264, organophosphorus compounds such as fenitrothion [O,O-dimethyl O-(3-methyl-4-nitrophenyl) phosphorothioate], fenthion [O,O-dimethyl O-(3-methyl-4-(methythio)phenyl) phosphorothioate], diazinon [O,O-diethyl O-2-isopropyl-6-methylpyrimidin-4-yl phosphorothioate], chlorpyrifos [O,O-diethyl O-3,5,6-trichloro-2-pyridyl phosphorothioate], DDVP [2,2-dichlorovinyl dimethyl phosphate], cyanophos [O-4-cyanophenyl O,O-dimethyl phosphorothioate], dimethoate [O,O-dimethyl S—(N-methylcarbamoylmethyl) dithiophosphate], phenthoate [ethyl 2-dimethoxyphosphinothioylthio(phenyl)acetate], malathion [diethyl (dimethoxyphosphinothioylthio)succinate], and azinphos-methyl [S-3,4-dihydro-4-oxo-1,2,3-benzotriazin-3-ylmethyl O,O-dimethyl phosphorodithioate]; carbamate compounds such as BPMC (2-sec-butylphenyl methylcarbamate), benfracarb [ethyl N-[2,3-dihydro-2,2-dimethylbenzofuran-7-yloxycarbonyl (methyl)aminothio]-N-isopropyl-β-alaninate], propoxur [2-isopropoxyphenyl N-methylcarbamate] and carbaryl [1-naphthyl-N-methylcarbamate], methomyl [S-methyl-N-[(methylcarbamoyl)oxy]thioacetimidate]; pyrethroid compounds such as etofenprox [2-(4-ethoxyphenyl)-2-methylpropyl-3-phenoxybenzyl ether], fenvalerate [(RS)-α-cyano-3-phenoxybenzyl (RS)-2-(4-chlorophenyl)-3-methylbutyrate], esfenvalerate [(S)-α-cyano-3-phenoxybenzyl (S)-2-(4-chlorophenyl)-3-methylbutyrate], fenpropathrin [(RS)-α-cyano-3-phenoxybenzyl 2,2,3,3-tetramethyl cyclopropane carboxylate], cypermethrin [(RS)-α-cyano-3-phenoxybenzyl (1RS)-cis,trans-3-(2,2-dichlorovinyl)-2,2-dimethylcyclopropanecarboxylate], permethrin [3-phenoxybenzyl (1RS)-cis,trans-3-(2,2-dichlorovinyl)-2,2-dimethylcyclopropanecarboxylate], cyhalothrin [(RS)-α-cyano-3-phenoxybenzyl (Z)-(1RS)-cis-3-(2-chloro-3,3,3-trifluoroprop-1-enyl)-2,2-dimethylcyclopropanecarboxylate], deltamethrin [(S)-α-cyano-3-phenoxybenzyl (1R)-cis-3-(2,2-dibromovinyl)-2,2-dimethylcyclopropanecarboxylate], cycloprothrin [(RS)-α-cyano-3-phenoxybenzyl (RS)-2,2-dichloro-1-(4-ethoxyphenyl)cyclopropanecarboxylate], fluvalinate [α-cyano-3-phenoxybenzyl N-(2-chloro-α,α,α-trifluoro-p-tolyl)-D-valinate], bifenthrin [2-methylbiphenyl-3-ylmethyl (Z)-(1RS)-cis-3-(2-chloro-3,3,3-trifluoroprop-1-enyl)-2,2-dimethylcyclopropanecarboxylate], 2-methyl-2-(4-bromodifluoromethoxyphenyl)propyl 3-phenoxybenzyl ether, tralomethrin [(S)-α-cyano-3-phenoxybenzyl (1R-cis)-3-{(1RS)(1,2,2,2-tetrabromoethyl)}-2,2-dimethylcyclopropanecarboxylate], silafluofen [(4-ethoxyphenyl) {3-(4-fluoro-3-phenoxyphenyl)propyl}dimethylsilane], d-phenothrin [3-phenoxybenzyl (1R-cis,trans)-chrysanthemate], cyphenothrin [(RS)-α-cyano-3-phenoxybenzyl (1R-cis,trans)-chrysanthemate], d-resmethrin [5-benzyl-3-furylmethyl (1R-cis,trans)-chrysanthemate], acrinathrin [(S)-α-cyano-3-phenoxybenzyl (1R,cis(Z))-2,2-dimethyl-3-{3-oxo-3-(1,1,1,3,3,3-hexafluoropropyloxy)propenyl}cyclopropanecarboxylate], cyfluthrin [(RS)-α-cyano-4-fluoro-3-phenoxybenzyl 3-(2,2-dichlorovinyl)-2,2-dimethylcyclopropanecarboxylate], tefluthrin [2,3,5,6-tetrafluoro-4-methylbenzyl (1RS-cis(Z))-3-(2-chloro-3,3,3-trifluoroprop-1-enyl)-2,2-dimethylcyclopropanecarboxylate], transfluthrin [2,3,5,6-tetrafluorobenzyl (1R-trans)-3-(2,2-dichlorovinyl)-2,2-dimethylcyclopropanecarboxylate], tetramethrin [3,4,5,6-tetrahydrophthalimidomethyl (1RS)-cis,trans-chrysanthemate], allethrin [(RS)-3-allyl-2-methyl-4-oxocyclopent-2-enyl (1RS)-cis,trans-chrysanthemate], prallethrin [(S)-2-methyl-4-oxo-3-(2-propynyl)cyclopent-2-enyl (1R)-cis,trans-chrysanthemate], empenthrin [(RS)-1-ethynyl-2-methyl-2-pentenyl (1R)-cis, trans-chrysanthemate], imiprothrin [2,5-di oxo-3-(prop-2-ynyl)imidazolidin-1-ylmethyl (1R)-cis,trans-2,2-dimethyl-3-(2-methyl prop-1-enyl)cyclopropanecarboxylate], d-furamethrin [5-(2-propynyl)furfuryl (1R)-cis,trans-chrysanthemate] and 5-(2-propynyl)furfuryl 2,2,3,3-tetramethyl cyclopropanecarboxylate; nitroimidazoliiine derivatives such as imidacioprid (1-(6-chloro-3-pyridylmethyl)-N-nitroimidazolidin-2-ylideneamine); N-cyanoamidine derivatives such as N-cyano-N′-methyl-N′-(6-chloro-3-pyridylmethyl)acetamidine; nitenpyram [N-(6-chloro-3-pyridylmethyl)-N-ethyl-N-methyl-2-nitrovynylidenediamine]; thiacloprid [1-(2-chloro-5-pyridylmethyl)-2-cyanoiminothiazoline]; thiamethoxam [3-((2-chloro-5-thiazolyl) methyl)-5-methyl-4-nitroiminotetrahydro-1,3,5-oxadiazine]; 1-methyl-2-nitro-3-((3-tetrahydrofuryl)methyl)guanidine; 1-(2-chloro-5-thiazolyl)methyl-3-methyl-2-nitroguanidine; nitroiminohexahydro-1,3,5-triazine derivatives; chlorinated hydrocarbons such as endosulfan [6,7,8,9,10,10-hexachloro-1,5,5a,6,9,9a-hexahydro-6,9-methano-2,4,3-benzodioxathiepine oxide], γ-BHC [1,2,3,4,5,6-hexachlorocyclohexane] and 1,1-bis(chlorophenyl)-2,2,2-trichloroethanol; benzoylphenylurea compounds such as chlorfluazuron [1-(3,5-dichloro-4-(3-chloro-5-trifluoromethylpyridyn-2-yloxy)phenyl)-3-(2,6-di fluorobenzoyl)urea], teflubenzuron [1-(3,5-dichloro-2,4-di fluorophenyl)-3-(2,6-di fluorobenzoyl)urea] and flufenoxuron [1-(4-(2-chloro-4-trifluoromethyl phenoxy)-2-fluorophenyl)-3-(2,6-di fluorobenzoyl)urea]; juvenile hormone like compounds such as pyriproxyfen [4-phenoxyphenyl 2-(2-pyridyloxy)propyl ether], methoprene [isopropyl (2E,4E)-11-methoxy-3,7,11-trimethyl-2,4-dodecadienoate] and hydroprene [ethyl (2E,4E)-11-methoxy-3,7,11-trimethyl-2,4-dodecadienoate]; thiourea derivatives such as diafenthiuron [N-(2,6-diisopropyl-4-phenoxyphenyl)-N′-tert-butylcarbodiimide]; phenylpyrazole compounds; 4-bromo-2-(4-chlorophenyl)-1-ethoxymethyl-5-trifluoromethylpyrrol-3-carbonitrile [chlorfenapil]; metoxadiazone [5-methoxy-3-(2-methoxyphenyl)-1,3,4-oxadiazol-2(3H)-one], bromopropylate [isopropyl 4,4-dibromobenzilate], tetradifon [4-chlorophenyl 2,4,5-trichlorophenyl sulfone], chinomethionat [S,S-6-methyl quinoxaline-2,3-diyldithiocarbonate], pyri dab en [2-tert-butyl-5-(4-tert-butylbenzylthio)-4-chloropyridazin-3(2H)-one], fenpyroximate [tert-butyl (E)-4-[(1,3-dimethyl-5-phenoxypyrazol-4-yl)methyleneaminooxymethyl]benzoate], tebufenpyrad [N-(4-tert-butylbenzyl)-4-chloro-3-ethyl-1-methyl-5-pyrazolecarboxamide], polynactins complex [tetranactin, dinactin and trinactin], pyrimidifen [5-chloro-N-[2-{4-(2-ethoxyethyl)-2,3-dimethylphenoxy}ethyl]-6-ethylpyrimidin-4-amine], milbemectin, abamectin, ivermectin, and azadirachtin [AZAD]. Examples of the repellants that may be synergistically combined with the disclosed pogostone derivatives include, but are not limited to 3,4-carane-diol, N,N-diethyl-m-toluamide, 1-methylpropyl 2-(2-hydroxyetnyl)-1-piperidinecarboxylate, p-menthane-3,8-diol and plant essential oil such as hyssop oil, and examples of further synergists include bis-(2,3,3,3-tetrachloropropyl) ether (S-421), N-(2-ethylhexyl)bicyclo [2.2.1]hept-5-ene-2,3-dicarboximide (MGK-264), and α-[2-(2-butoxy ethoxy)ethoxy]-4,5-methylenedioxy-2-propyltoluene (piperonyl butoxide). Similarly, disclosed herein are methods of applying the disclosed pogostone derivatives and salts thereof to a plant or animal in combination with known fungicides, antibiotics, anti-microbials, or herbicides.

### VI. Compositions Comprising the Disclosed Derivatives

Compositions suitable for use in the methods described herein can be formulated with one or more of the disclosed pogostone derivatives and salts thereof and an acceptable carrier or diluent. The content of the pogostone derivatives within a formulated composition may be from about 0.01 to about 95%. The pogostone derivatives may be formulated into various types of compositions, including but not limited to an oil solution, emulsifiable concentrate, wettable powder, flowable (aqueous suspension or aqueous emulsion), granule, dust and so on, by mixing with solid carrier, liquid carrier or gaseous carrier and optionally surfactant, the other formulation additive.

Non-limiting examples of solid carriers that can be used in a formulation comprising the disclosed pogostone derivatives include inorganic carriers such as clays (e.g., kaolin clay, diatomaceous earth, synthetic hydrated silicon oxide, bentonite, Fubasami clay, acid clay), talc, ceramics, sericite, quartz and calcium carbonate. Examples of the liquid carrier include water, alcohols (e.g., methanol, ethanol, higher alcohols), ketones (e.g., acetone, methyl ethyl ketone), aromatic hydrocarbons (e.g., benzene, toluene, xylene, ethylbenzene, methylnaphthalene), aliphatic hydrocarbons (e.g., hexane, cyclohexane, kerosene, gas oil), esters (ethyl acetate, butyl acetate), nitrites (e.g., acetonitrile, isobutyronitrile), ethers (e.g. diisopropyl ether, dioxane), acid amides (e.g., N,N-dimethylformamide, N,N-dimethyl acetamide), halogenated hydrocarbons (e.g., dichloromethane, trichloroethane, carbon tetrachloride), dimethyl sulfoxide and vegetable oils (e.g., soybean oil, cottonseed oil). Examples of the liquefied gaseous carrier include fluorocarbon, fluorohydrocarbon, LPG (liquefied petroleum gas), dimethyl ether and carbon dioxide.

Non-limiting examples of the surfactant optionally used in the disclosed formulations can include alkyl sulfate salts, alkylsulfonate salts, alkylarylsulfonate salts, alkyl aryl ethers, polyoxyethylenealkyl aryl ethers, polyethylene glycol ethers, polyhydric alcohol esters and sugar alcohol derivatives.

The other formulation auxiliaries are exemplified by sticking agents, dispersants, and stabilizers. Non-limiting examples of sticking agents and dispersants include casein, gelatin, polysaccharides (e.g., starch powder, gum arabic, cellulose derivatives, alginic acid), lignin derivatives, bentonite, sugars and synthetic water-soluble polymers (e.g., polyvinyl alcohol, polyvinylpyrrolidone, polyacrylic acids). Non-limiting examples of stabilizer include phenol type antioxidants such as BHT (2,6-di-tert-butyl-4-methyphenol) and BHA (mixture of 2-tert-butyl-4-methoxyphenol and 3-tert-butyl-4-methoxyphenol), amine type antioxidants such as diphenylamine, organic sulfur type antioxidants such as 2-mercaptobenzimidazole, PAP (acid isopropyl phosphate), vegetable oils, mineral oils, surfactants, fatty acids and esters of fatty acid.

Flowable formulations (aqueous suspension or aqueous emulsion) may comprise one or more of the disclosed pogostone derivatives, a dispersant, a suspension assistant (for example, protective colloid or a compound giving thixotropy), suitable auxiliaries (for example, antifoamer, rust preventive agent, stabilizer, developing agent, penetrating assistant, antifreezing agent, bactericide, fungicide, etc.) and water. Non-limiting examples of a protective colloid include gelatin, casein, gums, cellulose ethers and polyvinyl alcohol, and examples of the compound giving thixotropy include bentonite, aluminum magnesium silicate, xanthan gum and polyacrylic acids. Use of an oil, which can, in some instance, dissolve a disclosed pogostone derivative, in place of water can give suspension-in-oil formulation.

The formulations of emulsifiable concentrate, wettable powder, flowable and so on obtained above may be diluted with water or another suitable vehicle, and applied at 0.1 to 10000 ppm of the concentration of the pogostone derivative. The formulations of oil solution, granule, dust and so on are may be applied to an intended plant, seed, or animal directly as they are.

In some embodiments, a mixture of one or more of the disclosed pogostone derivatives or a liquid formulation thereof and a propellant can be charged into a pressure container with a spray nozzle to afford an aerosol of the disclosed controlling agents. Further, the disclosed pogostone derivatives or a liquid formulation thereof can be impregnated into a base material of mosquito-coil, mosquito-mat, ceramic board and so on to afford a heating volatile formulation such as mosquito-coil and mosquito-mat for electric heater; a heating fumigant formulation such as self-combustible fumigant, chemical reaction type fumigant and porous ceramic board fumigant; a non-heating volatile formulation such as resin volatile formulation and paper volatile formulation; a smoking formulation such as fogging; and an ULV formulation of the disclosed controlling agents. Furthermore, a liquid formulation of one or more of the disclosed pogostone derivatives can be charged into a container with an absorptive wick in the upper part to afford a bottle containing insecticidal liquid for volatilization by heating the absorptive wick.

Non-limiting examples of the propellant for aerosols include propane, butane, isobutane, dimethyl ether, methyl ethyl ether and methylal.

An example of the base material of the mosquito-coil is a mixture of raw plant powder such as wood powder and Pyrethrum marc and a binding agent like Tabu powder (powder of Machilus thunbergii), starch or gluten.

Examples of the base material of the mosquito-mat for electric heating fumigation include a plate of compacted fibrils of cotton linters and a mixture of pulp and cotton linters.

The base material of the self-combustible fumigant includes, for example, an exothermic agent (e.g., nitrate, nitrite, guanidine salt, potassium chlorate, nitrocellulose, ethylcellulose, wood powder), a pyrolytic stimulating agent (e.g., alkali metal salt, alkaline earth metal salt, dichromate, chromate), an oxygen source (e.g., potassium nitrate), a combustion assistant (e.g., melanin, wheat starch), a bulk filler (e.g., diatomaceous earth) and a binding agent (e.g., synthetic glue).

The base material of the chemical reaction type fumigant includes, for example, an exothermic agent (e.g., alkali metal sulfide, polysulfide, hydrogensufide and hydrated salt, calcium oxide), a catalytic agent (e.g., carbonaneous substance, iron carbide, activated clay), an organic foaming agent (e.g., azodicarbonamide, benzenesulfonylhydrazide, dinitrosopentamethylenetetramine, polystyrene, polyurethane, etc.) and a filler (e.g., natural or synthetic fibers).

An example of the base material of the resin volatile formulation is thermoplastic resin, and examples of the base material of the paper volatile formulation include filter paper and Japanese paper.

The disclosed pogostone derivatives may be combined with other known insecticides, herbicides, fungicides, antibiotics, anti-microbial s, or arthropod repellents. For example, in some embodiments, in order to prepare a synergistic combination the disclosed pogostone derivatives may be combined with chlorfenapyr, piperonyl butoxide (PBO), S,S,S-tributyl phosphorotrithioate (DEF), N,N-diethyl-m-toluamide (DEET), permethrin, dibrom, thiamethoxam, enzyme inhibitors, essential oils (e.g., cedarwood oil), or insecticides and acaricides including, but not limited to, diethyl maleate (DEM), MGK-264, organophosphorus compounds such as fenitrothion [O,O-dimethyl O-(3-methyl-4-nitrophenyl) phosphorothioate], fenthion [O,O-dimethyl O-(3-methyl-4-(methythio)phenyl) phosphorothioate], diazinon [O,O-di ethyl O-2-isopropyl-6-methylpyrimidin-4-yl phosphorothioate], chlorpyrifos [O,O-diethyl O-3,5,6-trichloro-2-pyridyl phosphorothioate], DDVP [2,2-dichlorovinyl dimethyl phosphate], cyanophos [O-4-cyanophenyl O,O-dimethyl phosphorothioate], dimethoate [O,O-dimethyl S—(N-methylcarbamoylmethyl) dithiophosphate], phenthoate [ethyl 2-dimethoxyphosphinothioylthio(phenyl)acetate], malathion [diethyl (dimethoxyphosphinothioylthio)succinate], and azinphos-methyl [S-3,4-dihydro-4-oxo-1,2,3-benzotriazin-3-ylmethyl O,O-dimethyl phosphorodithioate]; carbamate compounds such as BPMC (2-sec-butylphenyl methylcarbamate), benfracarb [ethyl N-[2,3-dihydro-2,2-dimethylbenzofuran-7-yloxycarbonyl (methyl)aminothio]-N-isopropyl-β-alaninate], propoxur [2-isopropoxyphenyl N-methylcarbamate] and carbaryl [1-naphthyl-N-methylcarbamate], methomyl [S-methyl-N-[(methylcarbamoyl)oxy]thioacetimidate]; pyrethroid compounds such as etofenprox [2-(4-ethoxyphenyl)-2-methylpropyl-3-phenoxybenzyl ether], fenvalerate [(RS)-α-cyano-3-phenoxybenzyl (RS)-2-(4-chlorophenyl)-3-methylbutyrate], esfenvalerate [(S)-α-cyano-3-phenoxybenzyl (S)-2-(4-chlorophenyl)-3-methylbutyrate], fenpropathrin [(RS)-α-cyano-3-phenoxybenzyl 2,2,3,3-tetramethyl cyclopropane carboxylate], cypermethrin [(RS)-α-cyano-3-phenoxybenzyl (1RS)-cis,trans-3-(2,2-dichlorovinyl)-2,2-dimethyl cyclopropanecarboxylate], permethrin [3-phenoxybenzyl (1RS)-cis,trans-3-(2,2-dichlorovinyl)-2,2-dimethylcyclopropanecarboxylate], cyhalothrin [(RS)-α-cyano-3-phenoxybenzyl (Z)-(1RS)-cis-3-(2-chloro-3,3,3-trifluoroprop-1-enyl)-2,2-dimethyl cyclopropanecarboxylate], deltamethrin [(S)-α-cyano-3-phenoxybenzyl (1R)-cis-3-(2,2-dibromovinyl)-2,2-dimethylcyclopropanecarboxylate], cycloprothrin [(RS)-α-cyano-3-phenoxybenzyl (RS)-2,2-dichloro-1-(4-ethoxyphenyl)cyclopropanecarboxylate], fluvalinate [α-cyano-3-phenoxybenzyl N-(2-chloro-α,α,α-trifluoro-p-tolyl)-D-valinate], bifenthrin [2-methylbiphenyl-3-ylmethyl (Z)-(1RS)-cis-3-(2-chloro-3,3,3-trifluoroprop-1-enyl)-2,2-dimethyl cyclopropanecarboxylate], 2-methyl-2-(4-bromodifluoromethoxyphenyl)propyl 3-phenoxybenzyl ether, tralomethrin [(S)-α-cyano-3-phenoxybenzyl (1R-cis)-3-{(1RS)(1,2,2,2-tetrabromoethyl)}-2,2-dimethylcyclopropanecarboxylate], silafluofen [(4-ethoxyphenyl) {3-(4-fluoro-3-phenoxyphenyl)propyl}dimethyl silane], d-phenothrin [3-phenoxybenzyl (1R-cis,trans)-chrysanthemate], cyphenothrin [(RS)-α-cyano-3-phenoxybenzyl (1R-cis,trans)-chrysanthemate], d-resmethrin [5-benzyl-3-furylmethyl (1R-cis,trans)-chrysanthemate], acrinathrin [(S)-α-cyano-3-phenoxybenzyl (1R,cis(Z))-2,2-dimethyl-3-{3-oxo-3-(1,1,1,3,3,3-hexafluoropropyloxy)propenyl}cyclopropanecarboxylate], cyfluthrin [(RS)-α-cyano-4-fluoro-3-phenoxybenzyl 3-(2,2-dichlorovinyl)-2,2-dimethyl cyclopropanecarboxylate], tefluthrin [2,3,5,6-tetrafluoro-4-methylbenzyl (1RS-cis(Z))-3-(2-chloro-3,3,3-trifluoroprop-1-enyl)-2,2-dimethylcyclopropanecarboxylate], transfluthrin [2,3,5,6-tetrafluorobenzyl (1R-trans)-3-(2,2-dichlorovinyl)-2,2-dimethylcyclopropanecarboxylate], tetramethrin [3,4,5,6-tetrahydrophthalimidomethyl (1RS)-cis,trans-chrysanthemate], allethrin [(RS)-3-allyl-2-methyl-4-oxocyclopent-2-enyl (1RS)-cis,trans-chrysanthemate], prallethrin [(S)-2-methyl-4-oxo-3-(2-propynyl)cyclopent-2-enyl (1R)-cis,trans-chrysanthemate], empenthrin [(RS)-1-ethynyl-2-methyl-2-pentenyl (1R)-cis, trans-chrysanthemate], imiprothrin [2,5-di oxo-3-(prop-2-ynyl)imidazolidin-1-ylmethyl (1R)-cis,trans-2,2-dimethyl-3-(2-methyl prop-1-enyl)cyclopropanecarboxylate], d-furamethrin [5-(2-propynyl)furfuryl (1R)-cis,trans-chrysanthemate] and 5-(2-propynyl)furfuryl 2,2,3,3-tetramethyl cyclopropanecarboxylate; nitroimidazoliiine derivatives such as imidacioprid (1-(6-chloro-3-pyridylmethyl)-N-nitroimidazolidin-2-ylideneamine); N-cyanoamidine derivatives such as N-cyano-N′-methyl-N′-(6-chloro-3-pyridylmethyl)acetamidine; nitenpyram [N-(6-chloro-3-pyridylmethyl)-N-ethyl-N-methyl-2-nitrovynylidenediamine]; thiacloprid [1-(2-chloro-5-pyridylmethyl)-2-cyanoiminothiazoline]; thiamethoxam [3-((2-chloro-5-thiazolyl) methyl)-5-methyl-4-nitroiminotetrahydro-1,3,5-oxadiazine]; 1-methyl-2-nitro-3-((3-tetrahydrofuryl)methyl)guanidine; 1-(2-chloro-5-thiazolyl)methyl-3-methyl-2-nitroguanidine; nitroiminohexahydro-1,3,5-triazine derivatives; chlorinated hydrocarbons such as endosulfan [6,7,8,9,10,10-hexachloro-1,5,5a,6,9,9a-hexahydro-6,9-methano-2,4,3-benzodioxathiepine oxide], γ-BHC [1,2,3,4,5,6-hexachlorocyclohexane] and 1,1-bis(chlorophenyl)-2,2,2-trichloroethanol; benzoylphenylurea compounds such as chlorfluazuron [1-(3,5-dichloro-4-(3-chloro-5-trifluoromethylpyridyn-2-yloxy)phenyl)-3-(2,6-di fluorobenzoyl)urea], teflubenzuron [1-(3,5-dichloro-2,4-di fluorophenyl)-3-(2,6-di fluorobenzoyl)urea] and flufenoxuron [14442-chloro-4-trifluoromethyl phenoxy)-2-fluorophenyl)-3-(2,6-di fluorobenzoyl)urea]; juvenile hormone like compounds such as pyriproxyfen [4-phenoxyphenyl 2-(2-pyridyloxy)propyl ether], methoprene [isopropyl (2E,4E)-11-methoxy-3,7,11-trimethyl-2,4-dodecadienoate] and hydroprene [ethyl (2E,4E)-11-methoxy-3,7,11-trimethyl-2,4-dodecadienoate]; thiourea derivatives such as diafenthiuron [N-(2,6-diisopropyl-4-phenoxyphenyl)-N′-tert-butylcarbodiimide]; phenylpyrazole compounds; 4-bromo-2-(4-chlorophenyl)-1-ethoxymethyl-5-trifluoromethylpyrrol-3-carbonitrile [chlorfenapil]; metoxadiazone [5-m ethoxy-3-(2-m ethoxyphenyl)-1,3,4-oxadiazol-2(3H)-one], bromopropylate [isopropyl 4,4-dibromobenzilate], tetradifon [4-chlorophenyl 2,4,5-trichlorophenyl sulfone], chinomethionat [S,S-6-methyl quinoxaline-2,3-diyl dithiocarbonate], pyri dab en [2-tert-butyl-5-(4-tert-butylbenzylthio)-4-chloropyridazin-3(2H)-one], fenpyroximate [tert-butyl (E)-4-[(1,3-dimethyl-5-phenoxypyrazol-4-yl)methyleneaminooxymethyl]benzoate], tebufenpyrad [N-(4-tert-butylbenzyl)-4-chloro-3-ethyl-1-methyl-5-pyrazolecarboxamide], polynactins complex [tetranactin, dinactin and trinactin], pyrimidifen [5-chloro-N-[2-{4-(2-ethoxyethyl)-2,3-dimethylphenoxy}ethyl]-6-ethylpyrimidin-4-amine], milbemectin, abamectin, ivermectin, and azadirachtin [AZAD]. Examples of the repellents that may be synergistically combined with the disclosed pogostone derivatives include, but are not limited to 3,4-carane-diol, N,N-diethyl-m-toluamide, 1-methylpropyl 2-(2-hydroxyetnyl)-1-piperidinecarboxylate, p-menthane-3,8-diol and plant essential oil such as hyssop oil, and examples of further synergists include bis-(2,3,3,3-tetrachloropropyl) ether (S-421), N-(2-ethylhexyl)bicyclo [2.2.1]hept-5-ene-2,3-dicarboximide (MGK-264), and α-[2-(2-butoxy ethoxy)ethoxy]-4,5-methylenedioxy-2-propyltoluene (piperonyl butoxide). The pogostone derivative and the additional compound may be prepared as part of a mixture, such that the two (or more) compounds are applied together, or the pogostone derivatives and the additional compounds may be applied separately (e.g., a plant could be treated with a first solution comprising a pogostone derivative and a second solution comprising an additional compound) either at the same time or sequentially.

Further non-limiting examples of the insecticides that may be combined with the disclosed pogostone derivatives include organophosphorus compounds such as fenitrothion [O,O-dimethyl O-(3-methyl-4-nitrophenyl) phosphorothioate], fenthion [O,O-dimethyl O-(3-methyl-4-(methythio)phenyl) phosphorothioate], diazinon [O,O-diethyl O-2-isopropyl-6-methylpyrimidin-4-yl phosphorothioate], chlorpyrifos [O,O-diethyl O-3,5,6-trichloro-2-pyridyl phosphorothioate], DDVP [2,2-dichlorovinyl dimethyl phosphate], cyanophos [O-4-cyanophenyl O,O-di methyl phosphorothioate], dimethoate [O,O-di methyl S—(N-methylcarbamoylmethyl) dithiophosphate], phenthoate [ethyl 2-dimethoxyphosphinothioylthio(phenyl)acetate], malathion [diethyl (dimethoxyphosphinothioylthio)succinate], and azinphos-methyl [S-3,4-dihydro-4-oxo-1,2,3-benzotriazin-3-ylmethyl O,O-dimethyl phosphorodithioate]; carbamate compounds such as BPMC (2-sec-butylphenyl methylcarbamate), benfracarb [ethyl N-[2,3-dihydro-2,2-dimethylbenzofuran-7-yloxycarbonyl (methyl)aminothio]-N-isopropyl-β-alaninate], propoxur [2-isopropoxyphenyl N-methylcarbamate] and carbaryl [1-naphthyl-N-methylcarbamate], methomyl [S-methyl-N-[(methylcarbamoyl)oxy]thioacetimidate]; pyrethroid compounds such as etofenprox [2-(4-ethoxyphenyl)-2-methylpropyl-3-phenoxybenzyl ether], fenvalerate [(RS)-α-cyano-3-phenoxybenzyl (RS)-2-(4-chlorophenyl)-3-methylbutyrate], esfenvalerate [(S)-α-cyano-3-phenoxybenzyl (S)-2-(4-chlorophenyl)-3-methylbutyrate], fenpropathrin [(RS)-α-cyano-3-phenoxybenzyl 2,2,3,3-tetramethyl cyclopropanecarboxylate], cypermethrin [(RS)-α-cyano-3-phenoxybenzyl (1RS)-cis,trans-3-(2,2-dichlorovinyl)-2,2-dimethyl cyclopropanecarboxylate], permethrin [3-phenoxybenzyl (1RS)-cis,trans-3-(2,2-dichlorovinyl)-2,2-dimethylcyclopropanecarboxylate], cyhalothrin [(RS)-α-cyano-3-phenoxybenzyl (Z)-(1RS)-cis-3-(2-chloro-3,3,3-trifluoroprop-1-enyl)-2,2-dimethyl cyclopropanecarboxylate], deltamethrin [(S)-α-cyano-3-phenoxybenzyl (1R)-cis-3-(2,2-dibromovinyl)-2,2-dimethylcyclopropanecarboxylate], cycloprothrin [(RS)-α-cyano-3-phenoxybenzyl (RS)-2,2-dichloro-1-(4-ethoxyphenyl)cyclopropanecarboxylate], fluvalinate [α-cyano-3-phenoxybenzyl N-(2-chloro-α,α,α-trifluoro-p-tolyl)-D-valinate], bifenthrin [2-methylbiphenyl-3-ylmethyl (Z)-(1RS)-cis-3-(2-chloro-3,3,3-trifluoroprop-1-enyl)-2,2-dimethyl cyclopropanecarboxylate], 2-methyl-2-(4-bromodifluoromethoxyphenyl)propyl 3-phenoxybenzyl ether, tralomethrin [(S)-α-cyano-3-phenoxybenzyl (1R-cis)-3-{(1RS)(1,2,2,2-tetrabromoethyl)}-2,2-dimethylcyclopropanecarboxylate], silafluofen [(4-ethoxyphenyl) {3-(4-fluoro-3-phenoxyphenyl)propyl}dimethylsilane], d-phenothrin [3-phenoxybenzyl (1R-cis,trans)-chrysanthemate], cyphenothrin [(RS)-α-cyano-3-phenoxybenzyl (1R-cis,trans)-chrysanthemate], d-resmethrin [5-benzyl-3-furylmethyl (1R-cis,trans)-chrysanthemate], acrinathrin [(S)-α-cyano-3-phenoxybenzyl (1R,cis(Z))-2,2-dimethyl-3-{3-oxo-3-(1,1,1,3,3,3-hexafluoropropyloxy)propenyl}cyclopropanecarboxylate], cyfluthrin [(RS)-α-cyano-4-fluoro-3-phenoxybenzyl 3-(2,2-dichlorovinyl)-2,2-dimethyl cyclopropanecarboxylate], tefluthrin [2,3,5,6-tetrafluoro-4-methylbenzyl (1RS-cis(Z))-3-(2-chloro-3,3,3-trifluoroprop-1-enyl)-2,2-dimethylcyclopropanecarboxylate], transfluthrin [2,3,5,6-tetrafluorobenzyl (1R-trans)-3-(2,2-dichlorovinyl)-2,2-dimethylcyclopropanecarboxylate], tetramethrin [3,4,5,6-tetrahydrophthalimidomethyl (1RS)-cis,trans-chrysanthemate], allethrin [(RS)-3-allyl-2-methyl-4-oxocyclopent-2-enyl (1RS)-cis,trans-chrysanthemate], prallethrin [(S)-2-methyl-4-oxo-3-(2-propynyl)cyclopent-2-enyl (1R)-cis,trans-chrysanthemate], empenthrin [(RS)-1-ethynyl-2-methyl-2-pentenyl (1R)-cis, trans-chrysanthemate], imiprothrin [2,5-di oxo-3-(prop-2-ynyl)imidazolidin-1-ylmethyl (1R)-cis,trans-2,2-dimethyl-3-(2-methyl prop-1-enyl)cyclopropanecarboxylate], d-furamethrin [5-(2-propynyl)furfuryl (1R)-cis,trans-chrysanthemate] and 5-(2-propynyl)furfuryl 2,2,3,3-tetramethyl cyclopropanecarboxylate; nitroimidazoliiine derivatives such as imidacioprid (1-(6-chloro-3-pyridylmethyl)-N-nitroimidazolidin-2-ylideneamine); N-cyanoamidine derivatives such as N-cyano-N′-methyl-N′-(6-chloro-3-pyridylmethyl)acetamidine; nitenpyram [N-(6-chloro-3-pyridylmethyl)-N-ethyl-N-methyl-2-nitrovynylidenediamine]; thiacloprid [1-(2-chloro-5-pyridylmethyl)-2-cyanoiminothiazoline]; thiamethoxam [3-((2-chloro-5-thiazolyl) methyl)-5-methyl-4-nitroiminotetrahydro-1,3,5-oxadiazine]; 1-methyl-2-nitro-3-((3-tetrahydrofuryl)methyl)guanidine; 1-(2-chloro-5-thiazolyl)methyl-3-methyl-2-nitroguanidine; nitroiminohexahydro-1,3,5-triazine derivatives; chlorinated hydrocarbons such as endosulfan [6,7,8,9,10,10-hexachloro-1,5,5a,6,9,9a-hexahydro-6,9-methano-2,4,3-benzodioxathiepine oxide], γ-BHC [1,2,3,4,5,6-hexachlorocyclohexane] and 1,1-bis(chlorophenyl)-2,2,2-trichloroethanol; benzoylphenylurea compounds such as chlorfluazuron [1-(3,5-dichloro-4-(3-chloro-5-trifluoromethylpyridyn-2-yloxy)phenyl)-3-(2,6-di fluorobenzoyl)urea], teflubenzuron [1-(3,5-dichloro-2,4-di fluorophenyl)-3-(2,6-di fluorobenzoyl)urea] and flufenoxuron [14442-chloro-4-trifluoromethyl phenoxy)-2-fluorophenyl)-3-(2,6-di fluorobenzoyl)urea]; juvenile hormone like compounds such as pyriproxyfen [4-phenoxyphenyl 2-(2-pyridyloxy)propyl ether], methoprene [isopropyl (2E,4E)-11-methoxy-3,7,11-trimethyl-2,4-dodecadienoate] and hydroprene [ethyl (2E,4E)-11-methoxy-3,7,11-trimethyl-2,4-dodecadienoate]; thiourea derivatives such as diafenthiuron [N-(2,6-diisopropyl-4-phenoxyphenyl)-N′-tert-butylcarbodiimide]; phenylpyrazole compounds; 4-bromo-2-(4-chlorophenyl)-1-ethoxymethyl-5-trifluoromethylpyrrol-3-carbonitrile [chlorfenapil]; metoxadiazone [5-m ethoxy-3-(2-methoxyphenyl)-1,3,4-oxadiazol-2(3H)-one], bromopropylate [isopropyl 4,4-dibromobenzilate], tetradifon [4-chlorophenyl 2,4,5-trichlorophenyl sulfone], chinomethionat [S,S-6-methylquinoxaline-2,3-diyldithiocarbonate], pyri dab en [2-tert-butyl-5-(4-tert-butylbenzylthio)-4-chloropyridazin-3(2H)-one], fenpyroximate [tert-butyl (E)-4-[(1,3-dimethyl-5-phenoxypyrazol-4-yl)methyleneaminooxymethyl]benzoate], tebufenpyrad [N-(4-tert-butylbenzyl)-4-chloro-3-ethyl-1-methyl-5-pyrazolecarboxamide], polynactins complex [tetranactin, dinactin and trinactin], pyrimidifen [5-chloro-N-[2-{4-(2-ethoxyethyl)-2,3-dimethylphenoxy}ethyl]-6-ethylpyrimidin-4-amine], milbemectin, abamectin, ivermectin and azadirachtin [AZAD]. Examples of the repellents include 3,4-carane-diol, N,N-diethyl-m-toluamide, 1-methylpropyl 2-(2-hydroxyetnyl)-1-piperidinecarboxylate, p-menthane-3,8-diol and plant essential oil such as hyssop oil, and examples of the synergists include bis-(2,3,3,3-tetrachloropropyl) ether (S-421), N-(2-ethylhexyl)bicyclo[2.2.1]hept-5-ene-2,3-dicarboximide (MGK-264) and α-[2-(2-butoxy ethoxy)ethoxy]-4,5-methylenedioxy-2-propyltoluene (piperonyl butoxide).

In some embodiments, arthropod repellents comprising the disclosed pogostone derivatives and salts thereof, particularly those that are to be applied directly to an animal (e.g., a human), may be formulated as a spray, an aerosol, a lotion, a gel, a cream, or a balm. In some embodiments, the disclosed pogostone derivatives and salts thereof may be formulated as part of a fragrance, perfume, or cologne.

In some embodiments, rather than applying the disclosed pogostone derivatives and salts thereof to a specific plant or animal, the compounds may be applied (e.g., sprayed or otherwise dispersed) in a general target area where it is desirable to kill or repel arthropods. The target area may be, for example, a place where people or animals are congregating, a site of a known insect infestation, or a field where crops are being grown.

The application amount and concentration of the disclosed pogostone derivatives can be suitably designed according to the type of the formulations, time, place, and method of application, kind of target arthropod/fungus/bacteria/microbe/plant, and the type of use desired (e.g., insecticide versus insect repellent).

Multiple studies (as shown in the Examples section) have been performed that demonstrate that the disclosed pogostone derivatives or salts thereof are highly efficacious insecticides. Moreover, their utility is diverse. By producing both mortality 1 day after application and immediate knockdown, these compounds are promising active ingredients for insecticidal formulations. The disclosed derivatives were efficacious in topical applications, spray cup testing, leaf dip assays, baits, and larvicide explorations, demonstrating their potential in multiple distinct environments. It has also been demonstrated that some derivatives were more efficacious than natural pyrethrins on a pyrethroid-resistant strain of mosquito, thus highlighting the ability of these molecules at controlling insecticide-resistant pest populations.

The following examples illustrate the invention. It should be understood, however, that the invention is not to be limited to the specific conditions or details described in these examples. All printed publications referenced herein are specifically incorporated by reference.

### VII. Examples

### Example 1—Insecticidal Property Assays

Topical applications on adult, female mosquitoes were performed using a modified World Health Organization protocol (WHOPES 2006). Pyrone derivatives were dissolved in certified acetone at various concentrations that would yield between 5% and 95% mortality at 24 hours post-treatment. Adult female mosquitoes were anesthetized with CO₂ and quickly transferred to a petri dish surrounded by ice to prevent reanimation. A filter paper was placed at the bottom of the petri dish to absorb condensation and replaced with a new filter paper for each new compound tested. For each application, a 0.2-4, volume of solution was applied to the pronotum of each female mosquito using a 10-4, gastight Hamilton syringe, and treated mosquitoes were transferred to a 4-ounce cup with tulle placed on the top to prevent escape. Treated mosquitoes were then moved to an environmentally controlled incubator (27° C., 80% relative humidity, 16:8-hour light:dark cycle) for 24 hours, at which point mortality was recorded. Mortality at 24 hours was defined as the percentage of arthropods that showed no movement (ataxia) after being prodded with a camel hair brush. Percentage knockdown at 1-hour was also recorded.

TABLE 1
Mosquito Toxicity (24 hours)

| | | | | LD₅₀ (ug/g | 95% FL (ug/g | | |
|---|---|---|---|---|---|---|---|
| Treatment | n | Slope (SE) | | mosquito) | mosquito) | χ² (df) | |
| Rosemary Oil | 400 | 8.64 | (2.09) | 33000 | 31000-35000 | 29.6 | (14) |
| Patchouli Oil | 1100 | 1.62 | (0.31) | 1500 | 1000-2000 | 242.6 | (42) |
| Thymol | 525 | 5.6 | (1.14) | 3500 | 3000-4100 | 69.8 | (16) |
| Carvacrol | 725 | 3.11 | (0.42) | 3400 | 2600-4200 | 60.6 | (23) |
| Pogostone | 975 | 1.886 | (0.26) | 387.63 | 287.62-541.95 | 51.07 | (24) |
| Formula 4 | | | | Not Tested | | | |
| Formula 5 | | | | Not Tested | | | |
| Formula 7 | 125 | — | | >1,500 | — | — | |
| Formula 12 | 125 | — | | >1,500 | — | — | |
| Formula 8 | 125* | — | | >1,500 | — | — | |
| Formula 13 | 850 | 1.6726 | (0.46) | 118.96 | 50.9-174.18 | 125.04 | (27) |
| Formula 14 | 750 | 1.54 | (0.43) | 139 | 59-271.5 | 295.5 | (24) |
| Formula 15 | | | | Not Assigned | | | |
| Formula 20 | 125 | — | | >1,500 | — | — | |
| Formula 22 | 125 | — | | >1,500 | — | — | |
| Formula 16 | 125 | — | | >1,500 | — | — | |
| Formula 17 | 125 | — | | >1,500 | — | — | |
| Formula 21 | 325 | 1.87 | (0.62) | 534.4 | 129-1510 | 34.1 | (8) |
| Formula 11 | 125 | — | | >1,500 | — | — | |
| Formula 23 | 950 | 0.842 | (0.15) | 77.1 | 20.4-185.3 | 82.8 | (30) |
| Formula 9 | 125 | — | | >1,500 | — | — | |
| Formula 18 | — | — | | >1,500 | — | — | |
| Formula 25 | — | — | | >1,500 | — | — | |
| Formula 19 | 525 | 1.12 | (0.28) | 24.7 | 5.8-55.1 | 86.6 | (15) |
| Formula 24 | — | — | | >1,500 | — | — | |
| Formula 26 | — | — | | >1,500 | — | — | |
| Formula 10 | — | — | | >1,500 | — | — | |
| Formula 28 | — | — | | >1,500 | — | — | |
| Formula 27 | 525 | 0.82 | (0.21) | 28.8 | 0.5-145.7 | 51.4 | (18) |
| Formula 29 | 625 | 2.63 | (0.95) | 47.7 | 11.5-69.6 | 89.1 | (19) |
| Formula 30 | 500 | 4.2 | (0.5) | 45.6 | 39.6-50.5 | 18 | (14) |
| Formula 31 | — | — | | >1,500 | — | — | |
| Formula 32 | — | — | | >1,500 | — | — | |
| Formula 33 | — | — | | >1,500 | — | — | |
| Formula 34 | 225 | 1.44 | (0.28) | 111 | 23.7-252.3 | 12.7 | (6) |
| Formula 36 | 225 | 1.66 | (0.45) | 192.9 | 20.7-492.9 | 26 | (6) |
| Natural Pyrethrins | 850 | 2.015 | (0.31) | 6.21 | 4.48-8.55 | 98.47 | (27) |

TABLE 2
Mosquito Knockdown Properties (1 hour)

| | | | | KD₅₀ (ug/g | 95% FL (ug/g | | |
|---|---|---|---|---|---|---|---|
| Treatment | n | Slope (SE) | | mosquito) | mosquito) | χ² (df) | |
| Rosemary Oil | 575 | 4.34 | (1.16) | 21000 | 10000-27000 | 78.96 | (16) |
| Patchouli Oil | 1225 | 1.38 | (0.40) | 210 | 6.8-610 | 315.5 | (35) |
| Thymol | 525 | 6.2 | (0.78) | 1700 | 1500-1900 | 33.2 | (16) |
| Carvacrol | 725 | 4.8 | (0.71) | 2000 | 1600-2500 | 70.5 | (23) |
| Rosemary Oil | 575 | 4.34 | (1.16) | 21000 | 10000-27000 | 78.96 | (16) |
| Pogostone | 300 | 5.52 | (1.2133) | 18.4 | 18.6-24.1 | 29.2 | (8) |
| Formula 4 | — | — | | Not Tested | — | — | |
| Formula 5 | — | — | | Not Tested | — | — | |
| Formula 7 | 125 | — | | >1,500 | — | — | |
| Formula 12 | — | — | | >1,500 | — | — | |
| Formula 8 | — | — | | >1,500 | — | — | |
| Formula 13 | 850 | 1.05 | (0.21) | 182 | 87-396.8 | 106.8 | (27) |
| Formula 14 | 750 | — | | >1,500 | — | — | |
| Formula 17 | 125 | — | | >1,500 | — | — | |
| Formula 21 | 325 | — | | >1,500 | — | — | |
| Formula 11 | 125 | — | | >1,500 | — | — | |
| Formula 23 | 950 | 0.578 | (0.21) | 78.7 | 0.114-740 | 207.6 | (30) |
| Formula 9 | 125 | — | | >1,500 | — | — | |
| Formula 18 | — | — | | >1,500 | — | — | |
| Formula 25 | — | — | | >1,500 | — | — | |
| Formula 19 | 525 | 0.73 | (0.14) | 384.5 | 53.6-1483 | 58.5 | (15) |
| Formula 24 | — | — | | >1,500 | — | — | |
| Formula 26 | — | — | | >1,500 | — | — | |
| Formula 10 | — | — | | >1,500 | — | — | |
| Formula 28 | — | — | | >1,500 | — | — | |
| Formula 27 | — | — | | >1,500 | — | — | |
| Formula 29 | — | 0.72 | (0.31) | 613.1 | 79.6-NA | 131.1 | (19) |

| | | | | | |
|---|---|---|---|---|---|
| Formula 30 | — | — | >1,500 | — | — |
| Formula 31 | — | — | >1,500 | — | — |
| Formula 32 | — | — | >1,500 | — | — |
| Formula 33 | — | — | >1,500 | — | — |
| Formula 34 | — | — | >1,500 | — | — |
| Formula 36 | — | — | >1,500 | — | — |

Several of the compounds, such as Formulas 13, 14, 23, 29, 30, 34, and 36, possessed better insecticidal properties than natural pogostone, and others, such as Formulas 19 and 27, possessed better insecticidal properties at least as well as natural pyrethrins (Table 1). Similarly, at least Formula 23 possessed better knockdown properties than natural pogostone (Table 2). Accordingly, the disclosed pogostone derivatives possess useful insecticidal and knockdown properties.

This protocol was followed for house flies with some minor alterations. As shown in the table below, similar results were observed for house flies as compared to mosquitoes, with many of the disclosed derivatives possessing useful insecticidal properties. In particular, Formula 23 and Formula 30 were extremely effective at killing house flies.

TABLE 3
House Fly Toxicity

| Compound | N | LD50 (ug/g fly) |
|---|---|---|
| Thymol | 300 | 3820 |
| Formula 13 | 120 | 5950 |
| Formula 23 | 180 | 1039 |
| Formula 19 | 140 | 1495 |
| Formula 14 | 150 | >6,000 |
| Formula 29 | 110 | 1761 |
| Formula 30 | 190 | 645 |
| Formula 31 | 170 | >6,000 |
| Natural pyrethrum | 60 | 125 |

In house flies, derivatives that contained halogen substitutions on the benzo-ring were significantly more toxic to house flies than the other derivatives tested. This increase in toxicity among these halogenated derivatives compared to non-halogenated derivatives was more drastic in house flies than on mosquitoes.

The disclosed pogostone derivatives were also screened against a number of other arthropod pest species to demonstrate its broad toxicity against diverse arthropods and arthropods (Coleoptera, Diptera, Lepidoptera, Blattoidea, Aphididae, Arachnida: Trombidiformes). Testing one of the most promising derivatives, Formula 13, against two-spotted spider mites, soybean aphids, maize weevils, German cockroaches, and European corn borers demonstrated that this derivative was not only toxic to these species, but was more toxic than a commonly used natural insecticide, thymol.

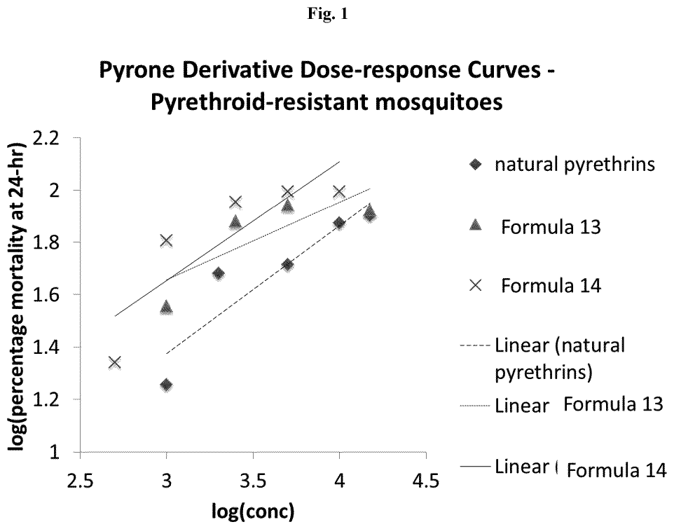

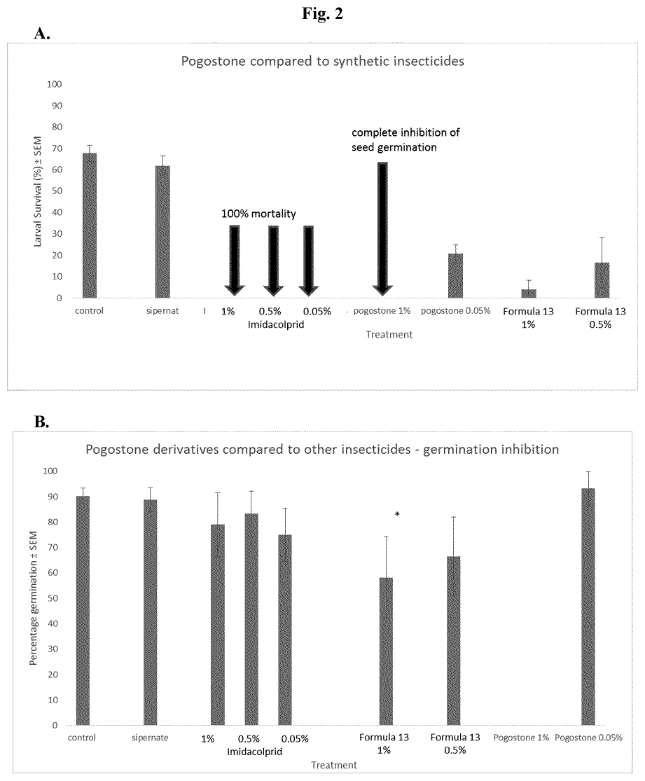

Of note, these data collectively indicate that knockdown at 1 hour was not related to mortality at 24 hours. These differences might indicate that immediate immobilization (knockdown) is mediated by action at a distinct site of action than the one that causes significant mortality. Alternatively, differences in toxicokinetics and breakdown might explain the two different bioactivity phenomena. This work demonstrates that the hydroxyl moiety associated with the pyrone ring are important molecular features for toxicity and fast immobilization. More derivatives have since been tested and certain substitutions are the most appropriate for insecticidal character. In general, shorter chain alkyl moieties a to the carbonyl adjacent to the pyrone moiety causes molecules with lower LD₅₀ values. Also, substitutions on the benzo-ring cause greater levels of toxicity, especially when those substitutions are halogens. Indeed, some of the most toxic compounds screened previously were exposed to a pyrethroid-resistant strain of Aedes aegypti. This strain was obtained from the wild in Puerto Rico and is maintained in culture by the Centers for Disease Control (CDC). It represents a strain that is resistant to pyrethroids via a target site mutation in the voltage-gated sodium channel. From this exploration, Formulas 13 and 14 outperformed natural pyrethrins against this strain. Dose response curves showing the results of these experiments are provided in FIG. 1. These results indicate that pogostone derivative toxicity is mediated via a distinct mechanism of action compared to natural pyrethrins. Moreover, it suggests that these molecules have potential in combatting pyrethroid-resistant arthropod pest populations. Pyrethroid resistance in wild populations of arthropod pests represents one of the most significant hurdles facing the effective control of pests in urban, agricultural, and medical and veterinary pest control.

### Example 2—Larvicidal Property Assays

Mosquito larvicide bioassays were performed by introducing various concentrations of test compounds (pyrone derivatives and rotenone) into acetone at various concentrations. 200 uL of these solutions were added to individual cups containing 10, 3-4 instar Aedes aegypti larvae and 25 mL of DI water. 2 mg of Tetramin™ was provided to each cup as a food source. Larval mortality was observed at 24 hours after introducing compounds. Ataxia was used as the indicator of larval mortality. At least 5 concentrations were used which caused between 10 and 90% mortality. LC₅₀ values were calculated using a PROC PROBIT model in SAS 9.4.

TABLE 4
Larvicidal Properties

| | | LC₅₀ | | | |
|---|---|---|---|---|---|
| Treatment | n | (μg/mL) | 95% CI | χ² | |
| Rotenone | 160 | 0.35 | 0.05-1.56 | 27.7 | (12) |
| Pogostone | 130 | 18.3 | 11.2-23.6 | 14.3 | (9) |
| Pogostone C (patented) | 130 | 20.3 | 4.9-26.7 | 17.5 | (9) |
| Formula 12 | 130 | 82.3 | 49.7-226 | 9.45 | (9) |
| Formula 13 | 220 | 1.34 | 0.49-2.5 | 36.8 | (18) |
| Formula 14 | 120 | 0.9 | 0.1-17.3 | 33.8 | (8) |
| Formula 28 | 100 | 9.16 | NA | 9.57 | (7) |

Several of the compounds, such as Formulas 13, 14, and 28, possessed better larvicidal properties than natural pogostone, and Formulas 13 and 14 also possessed better larvicidal properties than rotenone.

Aquatic environments represent a distinct environment compared to topical applications, and the efficacy of some of the more promising pogostone derivatives were explored in this series of tests. Again, Formula 13 appeared among the most successful analogs with Formula 14 being slightly more efficacious, however this difference between 13 and 14 was not statistically significant. Most other pogostone derivatives performed similarly to one another with LC₅₀ values of 17.3-82.3 ppm. Formulas 13 and 14 performed as well as the rotenone, the natural toxin chosen as a comparison. This data demonstrates the potential of these derivatives as potent insecticides/larvicides.

### Example 3—Seed Treatment Testing

Corn seeds obtained were untreated and did not contain any form of pesticide coating. Corn seeds used were a non-transgenic type strain of corn to prevent confounding results. Germination typically occurred between 2-4 days after moistening.

Seed coating was accomplished by placing 20 g of corn seeds or soybeans into a container with approximately 1 mL of a 1:5 Elmer's glue:water solution with a small amount of Triton-X100™ (present 100 μL/5 mL). For solid compounds, 0.25 g of compound was subsequently introduced into the container. This mixture of seeds, Elmer's Glue/water/Triton-X 100™ solution, and active compound was mixed thoroughly until seeds were coated with the active ingredient.

For each treatment, 0.25 g of liquid compound was dissolved in 5 mL of hexane. 0.25 g of Hi-Sil 233 silica gel was introduced into this mixture of hexane and active ingredient. The solvent was blown-off using a rotary evaporator, allowing for the compound to adsorb to the silica gel. This 0.5 g of silica gel:active ingredient was then used as the solid material used for coating the seeds. The seeds were then coated with the same method used for solid compound.

Three corn seeds coated in pogostone derivative treatments were germinated in small, 2-oz. Solo® condiment cups with lids. Germination of seeds was performed by carefully applying 500 μL of de-ionized water in order to fully surround each seed with water at the bottom of the cup. Parafilm® wax was placed over the top of the cup to maintain moisture and encourage germination of the seeds. Cups containing moistened seeds were allowed to germinate in the dark for 3 days before the start of the assay. Six live neonatal Western corn rootworm larvae were carefully applied to the roots of germinated corn seeds for each cup. After applying the larvae to the roots, 30 g of dried soil, sieved with a 600-μm FisherBrand sieve and moistened with 4.5 mL of water, was applied evenly throughout the cup. Organza was placed over the top of the cup and fastened in place with a cup lid. Cups were incubated at 28° C. in 40-50% humidity for 7 days. Surviving corn rootworm were enumerated and the average percentage survival was reported. A minimum of 4 replicates were performed for each treatment. Germination of seedlings was also recorded at the end of the assay by counting the total number of seeds that produced roots out of the 3 seeds in each container/replicate and recorded as total percentage germination. This was averaged across all the replicates of the assay.

The results shown in FIG. 2. As indicated in FIG. 2A, significant western corn rootworm mortality was observed when seeds were treated with pogostone or the derivative of Formula 13. However, as shown in FIG. 2B, pogostone also caused significant phytotoxicity with Formula 13 did not.

### Example 4—Spray-Cup Testing

10 adults of various arthropod species used for this characterization were placed into 8 oz. ice cream cups with tulle placed over the top to prevent the escape of arthropods from the container. Test formulations were added to spray bottles (MAINSTAYS™) and applied at the same distance and spray number to eliminate variation between manufacturer spray bottles. For all experiments, spray bottles were set to mist in order to prevent excess formulation from accumulating in the cups. Compounds were dissolved in water at various concentrations with an industrial emulsifier, and the toxicity of each concentration of compound was compared to 10% thymol solution and the emulsifier+water control. Mortality was recorded at 1 min, 5 min, 10 min, 15 min, 30 min, 4 hours, and 8 hours for each replicate. A minimum of 3 replicates of 10 arthropods/cup/replicate were performed for each formulation. Data was modeled using a logarithmic regression to show the speed-to-kill of each formulation and highlight differences among treatment groups. Different spray amounts were used for each arthropod, depending on susceptibility, e.g.:

House flies=8 sprays, 3 in

German roaches=10 sprays, 3 in

Maize weevils=8 sprays, 3 in

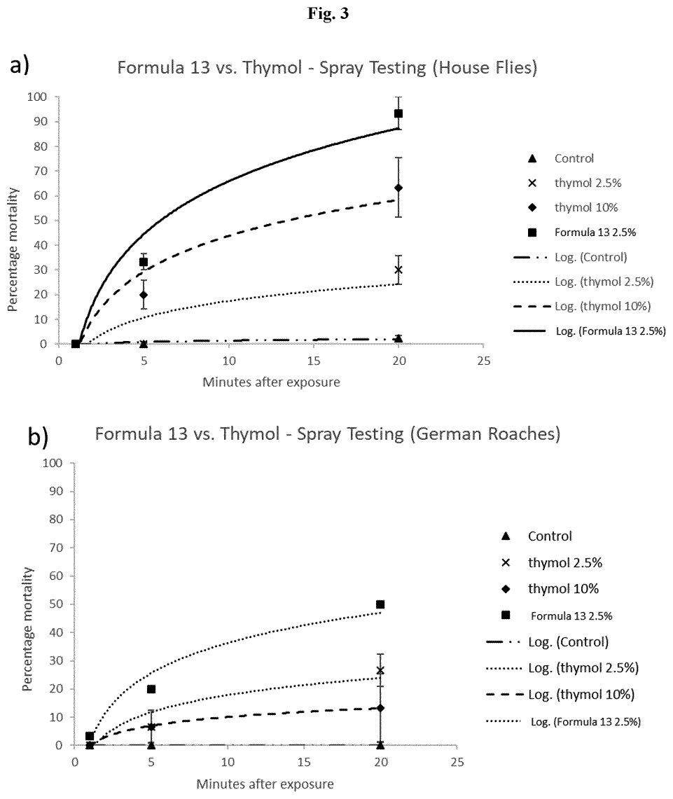

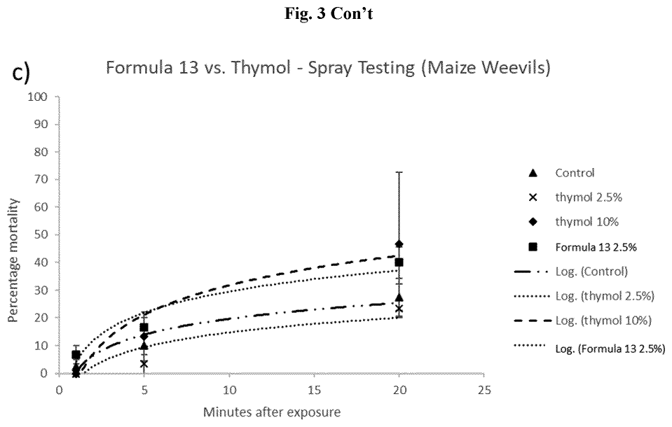

Mortality was assessed at different time points, as shown in FIG. 3. As can be seen in this figure, Formula 13 outperformed thymol at all time points across all arthropods tested.

### Example 5—Leaf Spray/Dip Testing

Soybean leaves were removed from a previously uninfested plant and dipped in solution containing water, various concentrations of compounds to be screened, and 0.5% industrial emulsifier. Leaves were allowed to air dry for 30 minutes before the beginning of the assay to assure excess formulation was not present on the treated leaves. Ten two-spotted spider mites or ten soybean aphid adults were gently placed on the top surface of the treated leaves. A minimum of three replicates were performed for each formulation tested in this experiment. Mortality of aphids or spider mites was recorded at 48 hours after their introduction to treated leaves. All treatments were compared to a water+emulsifier control and a thymol+emulsifier control. Data was presented as the average mortality across all replicates along with the standard error of the mean.

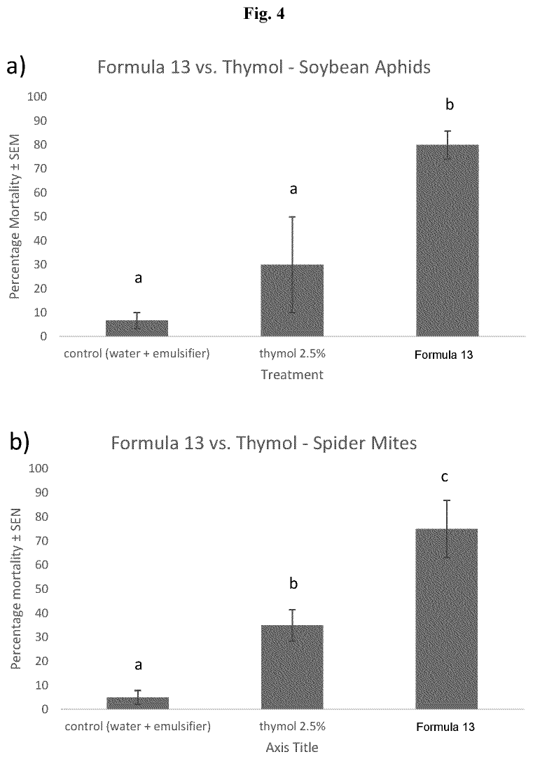

As shown in FIG. 4, Formula 13 performed better than thymol against both soybean aphids (FIG. 4a) and spider mites (FIG. 4b), resulting in significantly higher mortality.

### Example 6—House Fly Feeding Assay (Choice Assay)

Formulas 13, 17, 24, 26, 10, 27, and 29 and control (acetone and boric acid) were incorporated into 2 g of house fly food (1:1 dry milk and sucrose) at rate of 15%. Specifically, approximately 20-30 adult house flies ( ) were introduced into aquaria with a French square filled with de-ionized water. Cotton dental wicks were used to draw water up and present it to flies at the top of the French square. Compounds were introduced at a concentration of (15% w/w food) into 1:1 mixtures of evaporated milk powder and sucrose. MUSCALURE™, a potent house fly attractant/pheromone used in various house fly bait products, was also added to this mixture of bait and food at a concentration of 0.5% w/w food. Two grams of food mixed with compound was provided to house flies in each assay. Another 2 g of just fly food (no compound) was provided at the other end of the assay chamber to serve as untreated source of food. This was done to assess the ability of flies to feed on the treated food source (palatability). If acetone was used as the carrier throughout the experiment, the food was carefully stirred repeatedly to ensure evaporation so that no more acetone was present in the food. Total mortality was assessed at 5 days after introducing house flies into individual mason jars with food treated with different compounds.

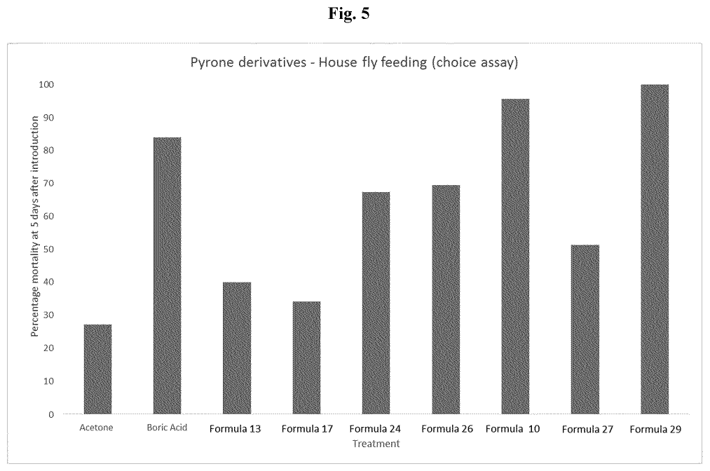

As shown in FIG. 5, the disclosed pogostone derivatives appear to be effective at killing house files and serving as a toxic bait.

Screening of select pogostone derivatives as insecticidal baits also demonstrated the toxicity of these compounds when supplied in an insecticidal bait matrix. Of the molecules tested, many outperformed boric acid, a commonly utilized toxicant found in some commercial insecticidal bait products. Formulas 10 and 29 performed better than boric acid applied at the same level, and Formula 24 and 26 performed similarly to boric acid. All of the pogostone derivatives screened caused at least some mortality in this assay compared to the control.

### Example 7—Potential to Synergize Other Insecticides

A low-dose concentration of Formula 13 (500 μg/mL) was applied in combination with chlorfenapyr (75 μg/mL), dibrom (10 μg/mL), thiamethoxam (100 μg/mL), and permethrin (2.5 μg/mL) to the pronotum of adult female Aedes aegypti in 0.2 μL of acetone. These concentrations were chosen as they produced little-to-no mortality at 24 hours after application. Knockdown (immobilization) was recorded at 1 hour after application and mortality was recorded at 24, 48, and 72 hours after initial application. These applications were run in parallel to a topical application of a combined mixture of 500 μg/mL Formula 13 and 75 μg/mL of chlorfenpayr to the pronotum of adult female mosquitoes. For this experiment, total of 25 mosquitoes were treated within each replicate, with a minimum of 3 replicates utilized for each treatment. Synergism was defined as the higher percentage mortality in the insecticidal combination of both Formula 13 and chlorfenapyr compared to each individual component applied by itself.

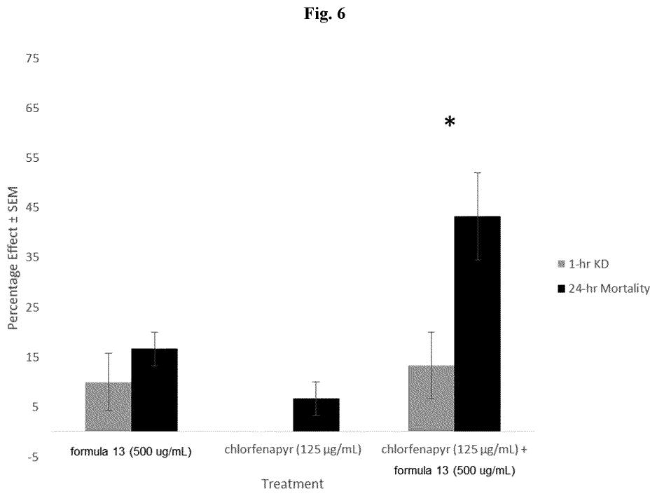

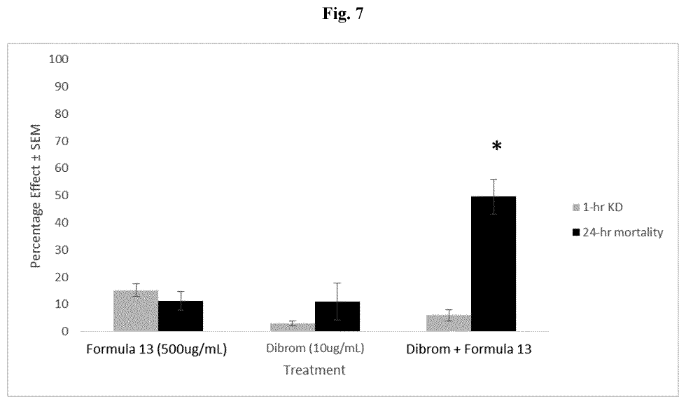

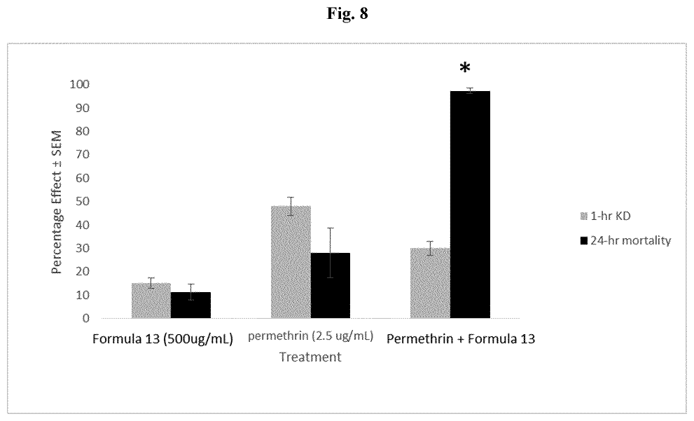

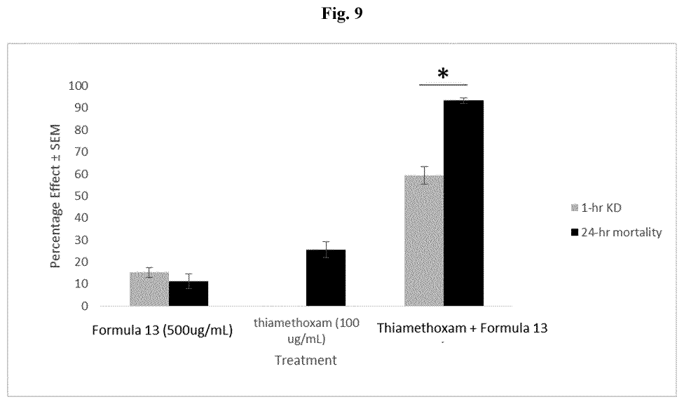

As shown in FIGS. 6-9, Formula 13 demonstrated an ability to enhance the efficacy of chlorfenapyr. Both Formula 13 and chlorfenapyr were applied individually and in combination at levels that produce limited mortality at 24 hours after application. At 24-hr after application, both Formula 13 and chlorfenapyr applied alone produced low levels of mortality. The combination of Formula 13 and chlorfenapyr, however, significantly increased the toxicity of each component compared to each component applied alone. Significant knockdown also occurred when Formula 13 was applied in combination with dibrom, permethrin, and thiamethoxam, respectively. Of the synthetic insecticides applied in combination with Formula 13, permethrin and thiamethoxam were the most significantly synergized by this pyrone derivative. Interestingly, Formula 13 also significantly increased the 1-hr knockdown of thiamethoxam, a neonicotinoid that typically displays low immediate knockdown potential when applied as a contact insecticide. Accordingly, the disclosed pogostone derivatives could be used to increase the efficacy of various slow-acting insecticides. By synergizing the lethality of various insecticides and also causing fast immobilization after application, these molecules could be important insecticidal additives or active ingredients in future insecticidal formulations.

Combinations of 2000 μg/mL Formula 13 and various detoxification enzyme inhibitors were applied to adult female mosquitoes in a volume of 0.2 μL to identify which detoxification pathway was most responsible for clearance of bioactive pogostone from the insect. A dose of 0.2 uL of either 1000 μg/mL of S,S,S-tributyl phosphorotrithioate (DEF) or 5000 μg/mL piperonyl butoxide (PBO) or 5000 μg/mL diethyl maleate (DEM) was applied 4 hours before a dose of 0.2 uL 2000 μg/mL of Formula 13 was applied to adult Aedes aegypti. This was done to significantly decrease the detoxification capacity of each of these enzyme systems. The concentrations of inhibitors were chosen due to their inability to cause toxicity on their own at this application rate. The concentration of Formula 13 was chosen as the approximate LD₂₅ of this compound against adult female Aedes aegypti mosquitoes. Combinations that caused statistically higher levels of mortality than pyrone 7 applied alone were considered informative. The goal of this exploration was to determine which processes might be most valuable in the detoxification of these derivatives.

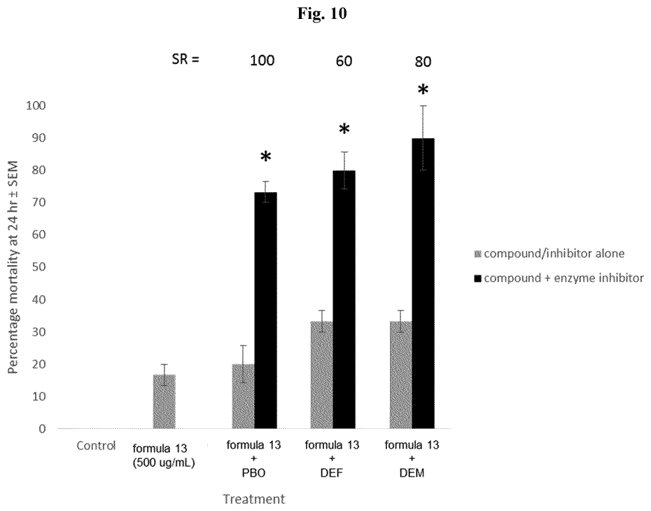

As shown in FIG. 10, the inhibition of monooxygenases (through the application of PBO) was the most significant for increasing the toxicity of Formula 13. This was followed by decreasing glutathione S-transferase activity (through the application of DEM) as the second-most important enzyme system in the detoxification of the pogostone derivative used in this study. The inhibition of esterases also significantly increased the toxicity of Formula 13. This indicates that combining Formula 13 and its related analogs with various detoxification inhibitors in future insecticidal formulations may be a promising lead in the development of highly efficacious insecticidal formulations. Moreover, this work suggests that molecules that are less susceptible to enzymatic degradation are also logical projections from these explorations on pogostone derivatives.

Combinations of select pogostone derivatives with select concentrations of PBO or plant essential oils was performed. For these explorations, topical applications on adult house flies were performed using a modified World Health Organization protocol (WHOPES 2006). Pogostone derivatives were dissolved in certified acetone at various concentrations that would yield between 5% and 95% mortality at 24 hours post-treatment. House flies were anesthetized with carbon dioxide and quickly transferred to a petri dish surrounded by ice to prevent reanimation. A filter paper was placed at the bottom of the petri dish to absorb condensation and replaced with a new filter paper for each new compound tested. For each application, a 0.5-μL volume of solution was applied to the pronotum of each house fly using a 25-μL gastight Hamilton syringe, and treated house flies were transferred to a 16-ounce mason jar with metal mesh to prevent the escape of house flies after reanimation. Synergists were applied as a subsequent application directly after the application of pogostone derivatives. Concentrations of synergists (plant oils or PBO) were chosen that produced no mortality at 24 hours when applied alone. Mortality at 24 hours was defined as the percentage of insects that showed no movement (ataxia) after being prodded with a camel hair brush (results not shown).

To assess the ability of PBO to increase the toxicity of a wide variety of pogostone derivatives, PBO and the pogostone derivatives were applied in combination to the pronotum of house flies. Table 5 highlights the LD₅₀ values of each pogostone derivative applied individually and in combination with PBO. This work allowed for the establishment of synergistic ratios for each compound to quantify the impact of applying each derivative in combination with PBO. Of all the pogostone derivatives tested, Formula 13 was the most significantly synergized by PBO, with a synergistic ratio of 17.8. A majority of pogostone derivatives were significantly synergized with a majority of SRs being above 2.

TABLE 5
Pogostone Derivatives Combined with PBO

| | N | LD₅₀ (μg/g) | SR |
|---|---|---|---|
| Formula 13 | 120 | 5950 | |
| Formula 13 + PBO | 150 | 334 | 17.8 |
| Formula 23 | 180 | 1039 | |
| Formula 23 + PBO | 150 | 385 | 2.82 |
| Formula 19 | 140 | 1495 | |
| Formula 19 + PBO | 150 | 705 | 2.12 |
| Formula 14 | 150 | >6,000 | |
| Formula 14 + PBO | 160 | 2143 | >2.79 |
| Formula 29 | 110 | 1761 | |
| Formula 29 + PBO | 130 | 1167 | 1.51 |
| Formula 30 | 190 | 645 | |
| Formula 30 + PBO | 80 | 153 | 4.22 |
| Formula 31 | 170 | >6,000 | |
| Formula 31 + PBO | 80 | 1100 | >5.45 |
| pyrethrum | 60 | 125 | |
| pyrethrum + PBO | 60 | 15.5 | 8.06 |

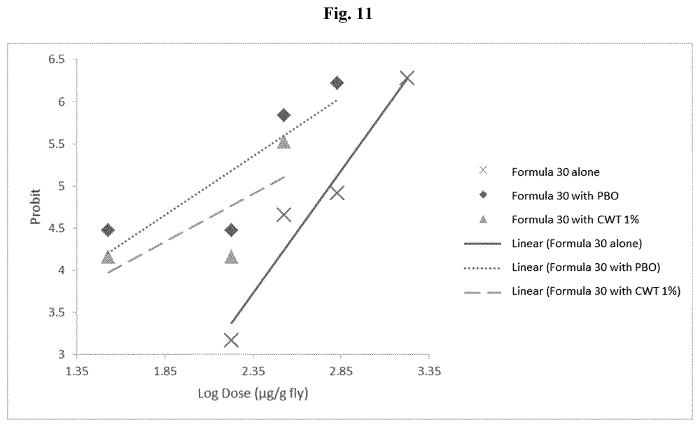

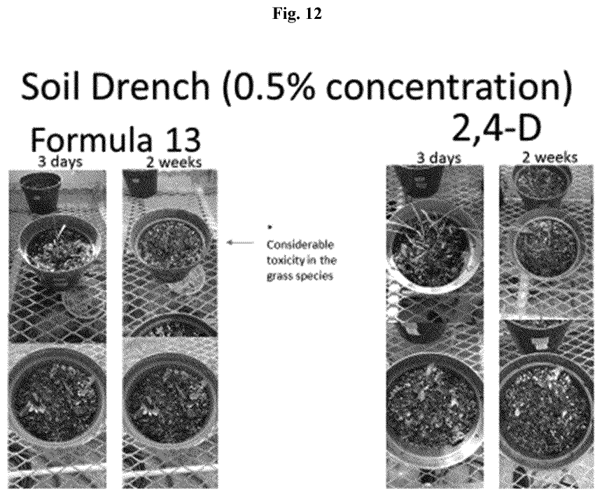

Finally, the potential of plant essential oils to synergize the effect of Formula 30 was assessed. Previous work has demonstrated that plant essential oils act to inhibit detoxification enzyme systems in insects. To characterize the degree to which this process enhanced the efficacy of Formula 30, this derivative was applied in combination with cedarwood (Texas type) oil, an oil previously shown to inhibit detoxification of detoxification processes. As shown in FIG. 11, a significant increase in toxicity was noted when applying Formula 30 in combination with both PBO and CWT. This highlights the potential of select plant oils to synergize the efficacy of Formula 30 and other pogostone derivatives in insecticidal formulations.

### Example 8—Herbicidal Properties

Dandelion (Taraxacum officinale) seeds were obtained from the Department of Agronomy at Iowa State University. Individual seeds were sown for each pot (6″ wide×4.5″ deep) with approximately 800 mL of soil for each plan. Only plants that germinated were used for the study. Plants were treated 1-2 weeks post emergence. Plants were watered every 2-3 days as needed. Giant foxtail (Setaria faberi) seeds were obtained from the Department of Agronomy at Iowa State University. Three seeds were sown for each pot (6″ wide×4.5″ deep) with approximately 800 mL of soil for each plan. Only plants that germinated were used for the study. Plants were treated 1-2 weeks post emergence. Plants were watered every 2-3 days as needed.

Formulations of the various active ingredients were created using the disclosed pogostone derivatives and Triton-X 100 to aid in the solvation of the active ingredient. Formulations consisted of 0.5% active ingredient by weight. Triton-X 100 was introduced into the formulation to aid in solubility at a final concentration of 0.25%. 40 mL of formulation (either 5% active ingredient) was applied to each pot containing approximately 3-5 plants per pot at 2-3 weeks after emergence. Observations were performed 3 days and 2 weeks after soil drench to determine the effects of the treatments on the various types and stages of plants. Phytotoxicity is reported as percentage of total plants treated compared to a control treatment that was exposed to a similar formulation (without the active monoterpenoid derivatives). Technical grade 2,4-dichlorophenoxyacetic acid (2,4-D) was used as the commercial comparison for phytotoxicity. For its solvation, potassium hydroxide was introduced at a 1:1 molar ratio with 2,4-D to ensure this molecule would adequately dissolve in water.

TABLE 6
Herbicidal Properties

| 3 days after soil drench - 40 mL of 0.5% solution | | | | 2 weeks after soil drench - 40 mL of 0.5% solution | | | |
|---|---|---|---|---|---|---|---|
| Plant | | | | Plant | | | |
| Species | Compound | Rep | % mortality | Species | Compound | Rep | % mortality |
| Foxtail | Formula 13 | 1 | 50% | Foxtail | Formula 13 | 1 | 100% |
| Foxtail | Formula 13 | 2 | 50% | Foxtail | Formula 13 | 2 | 100% |
| Foxtail | 2,4-D | 1 | 0% | Foxtail | 2,4-D | 1 | 66.70% |
| Foxtail | 2,4-D | 2 | 0.00% | Foxtail | 2,4-D | 2 | 50% |
| Dandelion | Formula 13 | 1 | 0% | Dandelion | Formula 13 | 1 | 0% |
| Dandelion | Formula 13 | 2 | 25% | Dandelion | Formula 13 | 2 | 25.00% |
| Dandelion | 2,4-D | 1 | 0% | Dandelion | 2,4-D | 1 | 100% |
| Dandelion | 2,4-D | 2 | 20.00% | Dandelion | 2,4-D | 2 | 80% |

Toxicity of various weed species exposed to a 0.5% solution of either Formula 13 or 2,4-D. In general, Formula 13 was more capable of producing mortality in foxtail than dandelions. 2,4-D caused the opposite effect, with dandelions being significantly more susceptible to this compound than Formula 13. These results indicate that the disclosed pogostone derivatives possess selective herbicidal properties that may be used to kill weeds and other undesirable plants.

### Example 9—Knockdown and Mortality of DHAA-Based Derivatives

The insecticidal properties, such as knockdown and mortality, were assessed for DHAA-based derivatives using methods similar to those described in Example 1 above. For example, adult female mosquitoes were anesthetized with CO₂ and quickly transferred to a petri dish surrounded by ice to prevent reanimation. A filter paper was placed at the bottom of the petri dish to absorb condensation and replaced with a new filter paper for each new compound tested. For each application, about 0.2-μL volume of solution was applied to the pronotum of each female mosquito using a syringe, and treated mosquitoes were transferred to a 4-ounce cup with tulle placed on the top to prevent escape. Treated mosquitoes were then moved to an environmentally controlled incubator (27° C., 80% relative humidity, 16:8-hour light:dark cycle) for 24 hours, at which point mortality was recorded. Mortality at 24 hours was defined as the percentage of arthropods that showed no movement (ataxia) after being prodded. Percentage knockdown at 1-hour was also recorded.

The first group of DHAA-based derivatives that were tested included Formula A2, Formula B3, Formula C₃, and Formula D3. A solution of 0.5% or 1.5% of each of these compounds was applied to the mosquitoes, as described above. Tables 7 and 8 describe the knockdown and mortality properties of these derivatives. Acetone was used as a control.

TABLE 7
Knockdown of First Generation DHAA-based Derivatives

| Compound | Percent Knockdown at 1 Hour |
|---|---|
| Formula A3 | 4 |
| Formula B3 | 2 |
| Formula C3 | 98 |
| Formula D3 | 6 |

Knockdown results are shown for 0.5% solutions

TABLE 8
Mortality of First Generation DHAA-based Derivatives

| Compound | Percent Mortality | SD | SEM |
|---|---|---|---|
| Acetone | 6 | 5 | 2 |
| Formula A3 at 0.5% | 5 | 2 | 1 |
| Formula A3 at 1.5% | 19 | 9 | 5 |
| Formula B3 at 0.5% | 9 | 8 | 5 |
| Formula B3 at 1.5% | 16 | 11 | 6 |
| Formula C3 at 0.5% | 89 | 6 | 4 |
| Formula C3 at 1.5% | 88 | 4 | 2 |
| Formula D3 at 0.5% | 15 | 6 | 4 |
| Formula D3 at 1.5% | 8 | 4 | 2 |

Of the compounds corresponding to Formulas A3, B3, C3, and D3, C3 performed the best in terms of highest percent knockdown and mortality. Accordingly, further derivatives with structures similar to Formula C3 were synthesized and tested for knockdown potential and mortality in house flies and mosquitoes using an experimental design similar to the one described above. These compounds include Formula C5, Formula C6, Formula C7, Formula C8, Formula C9, and Formula C10. The results for Formulas C6-C10 are shown in Table 9 below.

TABLE 9
Knockdown and Mortality of Second
Generation DHAA-based Derivatives

| | Formu- | Formu- | Formu- | Formu- | Formu- |
|---|---|---|---|---|---|
| | la C6 | la C7 | la C8 | la C9 | la C10 |
| House Fly Knockdown | 2.5% | 2.5% | 0% | 0% | 52.5% |
| at 0.5% concentration | | | | | |
| House Fly Mortality at | 10% | 13% | 3% | 5% | 23% |
| 0.5% concentration | | | | | |
| Mosquito Knockdown | 27% | 10% | 43% | 37% | 100% |
| at 0.5% concentration | | | | | |
| Mosquito Mortality at | 43% | 23% | 73% | 73% | 100% |
| 0.5% concentration | | | | | |
| House Fly Knockdown | 7% | 1% | 2% | 1% | 64% |
| at 1.5% concentration | | | | | |
| House Fly Mortality at | 6% | 9% | 13% | 6% | 24% |
| 1.5% concentration | | | | | |
| Mosquito Knockdown | 18% | 20% | 80% | 53% | 100% |
| at 1.5% concentration | | | | | |
| Mosquito Mortality at | 15% | 15% | 80% | 70% | 100% |
| 1.5% concentration | | | | | |

The DHAA-based derivatives that performed the best in the initial knockdown and mortality screenings were Formula C3, Formula C8, and Formula C10. The respective knockdown EC₅₀ values and mortality LD₅₀ values for these DHAA-based derivatives are compared with other disclosed posgostone derivatives, pogostone, natural pyrethrins, and other natural oils in Table 10 below.

TABLE 10
Comparison of LD₅₀ and EC₅₀ Values

| | 1-hour Knockdown EC₅₀ | 24-hour Mortality LD₅₀ |
|---|---|---|
| Compound | (μg/g mosquito) | (μg/g mosquito) |
| Pogostone | 95.2 | 262 |
| Formula 13 | 182 | 119 |
| Formula 14 | >1500 | 139 |
| Formula 23 | 78.7 | 77.1 |
| Formula 19 | 384.5 | 25 |
| Formula 27 | >1500 | 29 |
| Formula 39 | 55 | 157 |
| Formula 41 | >1500 | 105 |
| Formula C3 | N/A | 183 |
| Formula C8 | 661 | 256 |
| Formula C10 | 49 | 29 |
| Natural Pyrethrins | 1.65 | 6 |
| Rosemary Oil | Not calculated | 33000 |
| Patchouli Oil | Not calculated | 1500 |
| Thymol | Not calculated | 3500 |
| Carvacrol | Not calculated | 3400 |

Further DHAA-based derivatives were synthesized by varying the core structure of Formula C10. These “third generation” DHAA-based derivatives include Formulas C11, C12, C13, C14, C15, C16, C17, C18, C19, C20, C21, C22, C23, C34, C25, C26, and C27. The knockdown properties and mortality of Formulas C14-C27 were established in mosquitos using methods similar to those described above and shown in Table 11 below.

TABLE 11
Knockdown and Mortality of Third
Generation DHAA-based Derivatives

| | Knockdown | Knockdown | Mortality | Mortality |
|---|---|---|---|---|
| Compound | at 0.5% | at 1.5% | at 0.5% | at 1.5% |
| Formula C14 | 7% | 0% | 13% | 40% |
| Formula C15 | 23% | 50% | 33% | 63% |
| Formula C16 | 0% | 7% | 3% | 3% |
| Formula C17 | 27% | 13% | 47% | 7% |
| Formula C18 | 3% | 0% | 3% | 7% |
| Formula C19 | 83% | 83% | 83% | 77% |
| Formula C20 | 17% | 10% | 10% | 20% |
| Formula C21 | 3% | 7% | 0% | 0% |
| Formula C22 | 10% | 10% | 7% | 13% |
| Formula C23 | 10% | 10% | 13% | 27% |
| Formula C24 | 93% | 100% | 97% | 97% |
| Formula C25 | 93% | 100% | 100% | 100% |
| Formula C26 | 100% | 97% | 100% | 100% |
| Formula C27 | 100% | 73% | 97% | 83% |

Of the third generation DHAA-based derivatives, Formulas C15, C19, C24, C25, and C26 appear to be the most potent insecticidal compounds.

### Example 10—Contact Toxicity of DHAA-Based Derivatives

Filter papers were treated with 1 mL of the disclosed compounds in acetone at varying concentrations, and the filter papers were placed in a dish. Ten adult flies were added to each dish and a sugar pad was added to each dish to ensure that any mortality could be attributed to the compound instead of starvation. Mortality was assessed at 24 hours. Table 12 below provides the percent mortality results for the contact toxicity experiments.

TABLE 12
Contact Toxicity of Select Derivatives

| | 0.10% | 0.25% | 0.50% |
|---|---|---|---|
| Compound | Concentration | Concentration | Concentration |
| Natural Pyrethrins | N/A | 50% | N/A |
| Pogostone | 95% | 100% | 100% |
| Formula C10 | 15% | 0% | 45% |
| Formula C 19 | 0% | 0% | 0% |

### Example 11—Soybean Aphid Leaf Dip Assay for DHAA-Based Derivatives

Isolated soybean leaves were dipped into a pyrone solution containing either 0.5% or 1.5% of select DHAA-based derivatives. The leaves were allowed to dry for 30 minutes, and then place in a dish. Ten soybean aphids were placed on each leaf. Mortality was assessed at 24 hours.

At 24 hours, Formula C10 showed significant toxicity. Roughly 67% of soybean aphids exposed to the leaf dipped in 0.5% solution of Formula C10 were dead, and Roughly 90% of the soybean aphids exposed to the 1.5% solution were dead. More specifically, no live aphids were observed on the treated leaves and any surviving aphids were located on a separate section of the dish.

What is claimed is:

1. A pogostone derivative or salt thereof comprising the structure: 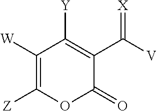
wherein V is selected from a group consisting of a substituted or unsubstituted C₁-C₁₂ unbranched or branched alkyl, substituted or unsubstituted C₂-C₁₂ unbranched or branched alkenyl, substituted or unsubstituted C₃-C₁₂ unbranched or branched alkynyl, substituted or unsubstituted C₃-C₁₂ unbranched or branched cycloalkyl, substituted or unsubstituted heteroaryl, and a substituted or unsubstituted C₃-C₁₂ unbranched or branched cycloalkenyl;
wherein W comprises a hydrogen or a substituted or unsubstituted C₃-C₆ saturated or unsaturated alkane or alkene;
wherein X is selected from a group consisting of an oxygen and a sulfur atom;
wherein Y is selected from a group consisting of a hydroxyl group, sulphhydryl, amino, a halogen, and an ether with variable alkyl length (C₁-C₇);
wherein Z comprises a substituted or unsubstituted C₁-C₁₂ unbranched or branched alkyl, substituted or unsubstituted C₃-C₁₂ unbranched or branched cycloalkyl, substituted or unsubstituted heteroaryl, and substituted or unsubstituted C₃-C₁₂ unbranched or branched cycloalkenyl; and
wherein W may be linked to Z via a substituted or unsubstituted C₃-C₆ saturated or unsaturated alkane or alkene.

2. The pogostone derivative of claim 1 wherein the structure is selected from a group consisting of Formula 1, Formula 2, Formula 3, Formula 4, Formula 5, Formula 6, Formula 7, Formula 8, Formula 9, Formula 10, Formula 11, Formula 12, Formula 13, Formula 14, Formula 15, Formula 16, Formula 17, Formula 18, Formula 19, Formula 20, Formula 21, Formula 22, Formula 23, Formula 24, Formula 25, Formula 26, Formula 27, Formula 28, Formula 29, Formula 30, Formula 31, Formula 32, Formula 33, Formula 34, Formula 35, Formula 36, Formula 37, Formula 38, Formula 39, Formula 40, Formula 41, Formula 42, Formula 43, Formula 44, Formula 45, Formula 46, Formula 47, and Formula 48.

3. A compound or salt thereof of the following formula: 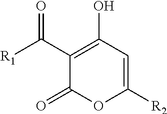
wherein R₁ is selected from a group consisting of a substituted or unsubstituted C₁-C₁₂ unbranched or branched alkyl, substituted or unsubstituted C₂-C₁₂ unbranched or branched alkenyl, substituted or unsubstituted C₃-C₁₂ unbranched or branched alkynyl, substituted or unsubstituted C₃-C₁₂ unbranched or branched cycloalkyl, substituted or unsubstituted heteroaryl, and a substituted or unsubstituted C₃-C₁₂ unbranched or branched cycloalkenyl; and
wherein R₂ is selected from a group consisting of a hydrogen, a substituted or unsubstituted C₃-C₁₂ unbranched or branched alkyl, substituted or unsubstituted C₂-C₁₂ unbranched or branched alkenyl, substituted or unsubstituted C₃-C₁₂ unbranched or branched alkynyl, substituted or unsubstituted C₃-C₁₂ unbranched or branched cycloalkyl, substituted or unsubstituted heteroaryl, and substituted or unsubstituted C₃-C₁₂ unbranched or branched cycloalkenyl, a hydroxyl group, sulphhydryl, amino, a halogen, and an ether with variable alkyl length (C₁-C₇).

4. The compound of claim 3, wherein R₁ a substituted or unsubstituted C₁-C₁₂ unbranched or branched alkyl.

5. The compound of claim 3, wherein R₂ is a methyl group.

6. The compound of claim 3, wherein R₂ comprises a unsubstituted C₃-C₁₂ unbranched or branched cycloalkyl.

7. The compound of claim 3, wherein the compound has the structure of Formula 4, Formula 5, or Formula 9.

8. A compound or salt thereof of the following formula: 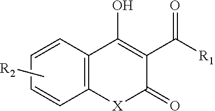
wherein R₁ is selected from a group consisting of a substituted or unsubstituted C₁-C₁₂ unbranched or branched alkyl, substituted or unsubstituted C₂-C₁₂ unbranched or branched alkenyl, substituted or unsubstituted C₃-C₁₂ unbranched or branched alkynyl, substituted or unsubstituted C₃-C₁₂ unbranched or branched cycloalkyl, substituted or unsubstituted heteroaryl, and a substituted or unsubstituted C₃-C₁₂ unbranched or branched cycloalkenyl; and
wherein R₂ is selected from a group consisting of a hydrogen, a substituted or unsubstituted C₃-C₁₂ unbranched or branched alkyl, substituted or unsubstituted C₂-C₁₂ unbranched or branched alkenyl, substituted or unsubstituted C₃-C₁₂ unbranched or branched alkynyl, substituted or unsubstituted C₃-C₁₂ unbranched or branched cycloalkyl, substituted or unsubstituted heteroaryl, and substituted or unsubstituted C₃-C₁₂ unbranched or branched cycloalkenyl, a hydroxyl group, sulphhydryl, amino, a halogen, and an ether with variable alkyl length (C₁-C₇);
and wherein X is selected from a group consisting of an oxygen, a carbon, and a nitrogen.

9. The compound of claim 8, wherein R₁ is a substituted or unsubstituted C₁-C₁₂ unbranched or branched alkyl.

10. The compound of claim 8, wherein R₂ is a hydrogen.

11. The compound of claim 8, wherein X is an oxygen.

12. The compound of claim 8, wherein X is a nitrogen.

13. The compound of claim 8, wherein the compound has the structure of Formula 13, Formula 14, Formula 16, Formula 17, Formula 19, Formula 20, Formula 21, Formula 22, Formula 23, Formula 24, or Formula 27.

14. The compound of claim 8, wherein the compound has the structure of Formula 13.

15. The compound of claim 8, wherein the compound has the structure of Formula 36, Formula 37, Formula 38, Formula 39, Formula 40, or Formula 41.

16. A compound or salt thereof of the following formula: 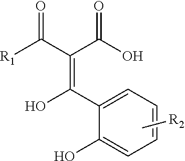
wherein R₁ is selected from a group consisting of a substituted or unsubstituted C₁-C₁₂ unbranched or branched alkyl, substituted or unsubstituted C₂-C₁₂ unbranched or branched alkenyl, substituted or unsubstituted C₃-C₁₂ unbranched or branched alkynyl, substituted or unsubstituted C₃-C₁₂ unbranched or branched cycloalkyl, substituted or unsubstituted heteroaryl, and a substituted or unsubstituted C₃-C₁₂ unbranched or branched cycloalkenyl; and
wherein R₂ is selected from a group consisting of a hydrogen, a substituted or unsubstituted C₃-C₁₂ unbranched or branched alkyl, substituted or unsubstituted C₂-C₁₂ unbranched or branched alkenyl, substituted or unsubstituted C₃-C₁₂ unbranched or branched alkynyl, substituted or unsubstituted C₃-C₁₂ unbranched or branched cycloalkyl, substituted or unsubstituted heteroaryl, and substituted or unsubstituted C₃-C₁₂ unbranched or branched cycloalkenyl, a hydroxyl group, sulphhydryl, amino, a halogen, and an ether with variable alkyl length (C₁-C₇).

17. A compound or salt having the structure of Formula 28.

18. A compound or salt thereof having a structure selected from the group consisting of Formula A3, Formula B3, Formula C3, Formula D3, Formula C5, Formula C6, Formula C7, Formula C8, Formula C9, Formula C10, Formula C11, Formula C12, Formula C13, Formula C14, Formula C15, Formula C16, Formula C17, Formula C18, Formula C19, Formula C20, Formula C21, Formula C22, Formula C23, Formula C24, Formula C25, Formula C26, Formula C27, Formula N1, Formula N2, Formula N3, Formula N4, Formula N5, Formula N6, Formula N7, Formula N8, Formula N9, Formula N10, AND Formula N11.

19. The compound or salt thereof of claim 18, wherein the structure is selected from the group consisting of Formula C10, Formula C15, Formula C19, Formula C24, Formula C25, and Formula C26.

20. An composition comprising the compound according to claim 1 and an acceptable vehicle.

21. The composition according claim 20, wherein the carrier is a solid, a liquid, or a gas.

22. The composition according to claim 20, wherein the composition is formulated as a lotion, spray, aerosol, or cream.

23. The composition according to claim 20, wherein the composition is formulated as a fragrance, perfume, or cologne.

24. A method of killing or repelling arthropods comprising applying to a target area an effective amount of a compound or salt thereof selected from the group consisting of: 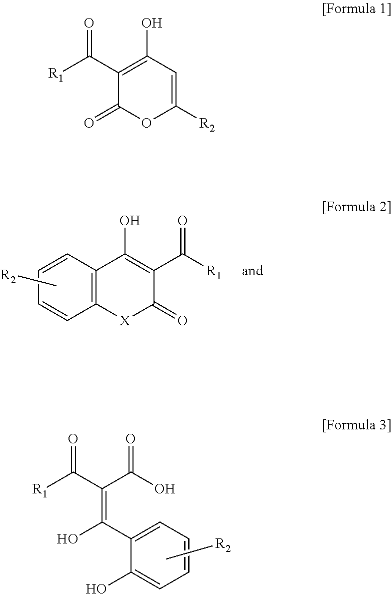
wherein R₁ is selected from a group consisting of a substituted or unsubstituted C₁-C₁₂ unbranched or branched alkyl, substituted or unsubstituted C₂-C₁₂ unbranched or branched alkenyl, substituted or unsubstituted C₃-C₁₂ unbranched or branched alkynyl, substituted or unsubstituted C₃-C₁₂ unbranched or branched cycloalkyl, substituted or unsubstituted heteroaryl, and a substituted or unsubstituted C₃-C₁₂ unbranched or branched cycloalkenyl;
wherein R₂ is selected from a group consisting of a hydrogen, a substituted or unsubstituted C₃-C₁₂ unbranched or branched alkyl, substituted or unsubstituted C₂-C₁₂ unbranched or branched alkenyl, substituted or unsubstituted C₃-C₁₂ unbranched or branched alkynyl, substituted or unsubstituted C₃-C₁₂ unbranched or branched cycloalkyl, substituted or unsubstituted heteroaryl, and substituted or unsubstituted C₃-C₁₂ unbranched or branched cycloalkenyl, a hydroxyl group, sulphhydryl, amino, a halogen, and an ether with variable alkyl length (C₁-C₇); and
wherein X is selected from a group consisting of an oxygen, a carbon, or a nitrogen.

25. The method of claim 24, wherein the compound has a structure selected from the group consisting of Formula 4, Formula 5, Formula 6, Formula 7, Formula 8, Formula 9, Formula 10, Formula 11, Formula 12, Formula 13, Formula 14, Formula 15, Formula 16, Formula 17, Formula 18, Formula 19, Formula 20, Formula 21, Formula 22, Formula 23, Formula 24, Formula 25, Formula 26, Formula 27, Formula 28, Formula 29, Formula 30, Formula 31, Formula 32, Formula 33, Formula 34, Formula 35, Formula 36, Formula 37, Formula 38, Formula 39, Formula 40, Formula 41, Formula 42, Formula 43, Formula 44, Formula 45, Formula 46, Formula 47, and Formula 48.

26. The method of claim 24, wherein the target area comprises a plant or a seed of a plant.

27. The method of claim 26, wherein the plant is a crop plant.

28. The method of claim 26, wherein the plant is selected from the group consisting of corn, soybeans, wheat, vegetables, fruits, or cotton.

29. The method of claim 24, wherein applying comprises spraying the target with the compound.

30. The method of claim 24, wherein applying comprises a vapor-delivery system.

31. The method of claim 24, wherein the target area comprises an animal

32. The method of claim 31, wherein the animal is a livestock animal or a companion animal.

33. The method of claim 31, wherein the animal is a human.

34. The method of claim 31, wherein applying comprises spraying the animal with a liquid or aerosol.

35. The method of claim 31, wherein applying comprises contacting the animal with a lotion, gel, cream, or balm comprising the compound.

36. The method of claim 24, wherein the arthropod is selected form the group consisting of mosquitos, ticks, fleas, ants, corn borers, grain borers, beetles, cockroaches, blood-sucking insects, biting insects, blackfly, house flies, barn fly, face fly, bush fly, deer fly, horse fly, gnats, beer bug, louse, bed bug, earwig, ant, aphid, spruce bud worm, sand flea, tsetse fly, assassin bug, biting flies, sand fly, stored grain pests, clothes moths, mites, spiders, phytophagous pests, and hematophagous pests.

37. A method of killing or repelling arthropods comprising applying to a target area an effective amount of a compound or salt thereof selected from the group consisting of Formula A3, Formula B3, Formula C3, Formula D3, Formula C5, Formula C6, Formula C7, Formula C8, Formula C9, Formula C10, Formula C11, Formula C12, Formula C13, Formula C14, Formula C15, Formula C16, Formula C17, Formula C18, Formula C19, Formula C20, Formula C21, Formula C22, Formula C23, Formula C24, Formula C25, Formula C26, Formula C27, Formula N1, Formula N2, Formula N3, Formula N4, Formula N5, Formula N6, Formula N7, Formula N8, Formula N9, Formula N10, and Formula N11.

38. The method of claim 37, wherein the target area comprises a plant or the seed of a plant.

39. The method of claim 38, wherein the plant is a crop plant.

40. The method of claim 38, wherein the plant is selected from the group consisting of corn, soybeans, wheat, vegetables, fruits, or cotton.

41. The method of claim 37, wherein applying comprises spraying the target with the compound.

42. The method of claim 37, wherein applying comprises a vapor-delivery system.

43. The method of claim 37, wherein the target area comprises an animal

44. The method of claim 43, wherein the animal is a livestock animal or a companion animal.

45. The method of claim 43, wherein the animal is a human.

46. The method of claim 43, wherein applying comprises spraying the animal with a liquid or aerosol.

47. The method of claim 43, wherein applying comprises contacting the animal with a lotion, gel, cream, or balm comprising the compound.

48. The method of claim 37, wherein the arthropod is selected form the group consisting of mosquitos, ticks, fleas, ants, corn borers, grain borers, beetles, cockroaches, blood-sucking insects, biting insects, blackfly, house flies, barn fly, face fly, bush fly, deer fly, horse fly, gnats, beer bug, louse, bed bug, earwig, ant, aphid, spruce bud worm, sand flea, tsetse fly, assassin bug, biting flies, sand fly, stored grain pests, clothes moths, mites, spiders, phytophagous pests, and hematophagous pests.

49. A method of killing or reducing the growth of undesirable vegetation or weeds comprising applying to a target area an effective amount of a compound or salt thereof selected from the group consisting of: 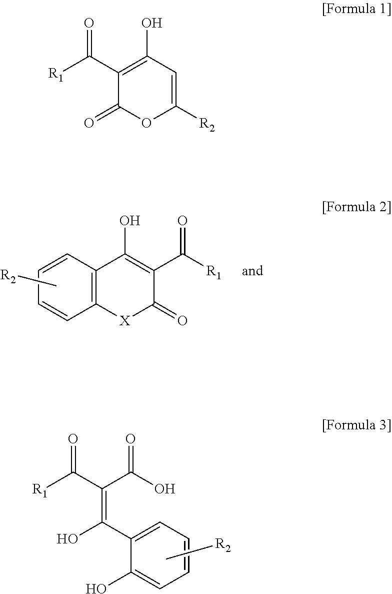
wherein R₁ is selected from a group consisting of a substituted or unsubstituted C₁-C₁₂ unbranched or branched alkyl, substituted or unsubstituted C₂-C₁₂ unbranched or branched alkenyl, substituted or unsubstituted C₃-C₁₂ unbranched or branched alkynyl, substituted or unsubstituted C₃-C₁₂ unbranched or branched cycloalkyl, substituted or unsubstituted heteroaryl, and a substituted or unsubstituted C₃-C₁₂ unbranched or branched cycloalkenyl;
wherein R₂ is selected from a group consisting of a hydrogen, a substituted or unsubstituted C₃-C₁₂ unbranched or branched alkyl, substituted or unsubstituted C₂-C₁₂ unbranched or branched alkenyl, substituted or unsubstituted C₃-C₁₂ unbranched or branched alkynyl, substituted or unsubstituted C₃-C₁₂ unbranched or branched cycloalkyl, substituted or unsubstituted heteroaryl, and substituted or unsubstituted C₃-C₁₂ unbranched or branched cycloalkenyl, a hydroxyl group, sulphhydryl, amino, a halogen, and an ether with variable alkyl length (C₁-C₇); and
wherein X is selected from a group consisting of an oxygen, a carbon, or a nitrogen.

50. The method of claim 49, wherein the compound has a structure selected from the group consisting of Formula 4, Formula 5, Formula 6, Formula 7, Formula 8, Formula 9, Formula 10, Formula 11, Formula 12, Formula 13, Formula 14, Formula 15, Formula 16, Formula 17, Formula 18, Formula 19, Formula 20, Formula 21, Formula 22, Formula 23, Formula 24, Formula 25, Formula 26, Formula 27, Formula 28, Formula 29, Formula 30, Formula 31, Formula 32, Formula 33, Formula 34, Formula 35, Formula 36, Formula 37, Formula 38, Formula 39, Formula 40, Formula 41, Formula 42, Formula 43, Formula 44, Formula 45, Formula 46, Formula 47, and Formula 48.

51. The method of any claim 50, wherein the undesirable vegetation comprises pigweed (Amaranthus retroflexus), poinsettia (Euphorbia heterophylla), nutsedge (Cyperus esculentus), morning glory (Ipomoea hederacea), or a combination thereof.

52. A method of killing or reducing the growth of undesirable vegetation or weeds comprising applying to a target area an effective amount of a compound or salt thereof selected from the group consisting of Formula A3, Formula B3, Formula C3, Formula D3, Formula C5, Formula C6, Formula C7, Formula C8, Formula C9, Formula C10, Formula C11, Formula C12, Formula C13, Formula C14, Formula C15, Formula C16, Formula C17, Formula C18, Formula C19, Formula C20, Formula C21, Formula C22, Formula C23, Formula C24, Formula C25, Formula C26, Formula C27, Formula N1, Formula N2, Formula N3, Formula N4, Formula N5, Formula N6, Formula N7, Formula N8, Formula N9, Formula N10, AND Formula N11.

53. The method of any claim 52, wherein the undesirable vegetation comprises pigweed (Amaranthus retroflexus), poinsettia (Euphorbia heterophylla), nutsedge (Cyperus esculentus), morning glory (Ipomoea hederacea), or a combination thereof.
